(19) United States
(12) Patent Application Publication
(10) Pub. No.: US 2025/0213523 A1
(43) Pub. Date: Jul. 3, 2025

(54) COMBINATION OF HDAC INHIBITORS AND STATINS FOR USE IN THE TREATMENT OF PANCREATIC CANCER

(71) Applicant: ISTITUTO NAZIONALE TUMORI IRCCS - FONDAZIONE G. PASCALE, Napoli (IT)

(72) Inventors: Alfredo BUDILLON, Napoli (IT); Maria Serena ROCA, Napoli (IT); Federica IANNELLI, Napoli (IT); Francesca BRUZZESE, Napoli (IT)

(73) Assignee: ISTITUTO NAZIONALE TUMORI IRCCS - FONDAZIONE G. PASCALE, Napoli (IT)

(21) Appl. No.: 18/853,315

(22) PCT Filed: Apr. 5, 2023
(86) PCT No.: PCT/EP2023/058948

§ 371 Date: Oct. 1, 2024

(30) Foreign Application Priority Data
Apr. 5, 2022 (EP) 22166770.2

(51) Int. Cl. A61K 31/366; A61K 31/167; A61K 31/19; A61K 31/4045; A61K 31/4406; A61K 45/06; A61P 35/00

(52) U.S. Cl. CPC A61K 31/366; A61K 31/167; A61K 31/19; A61K 31/4045; A61K 31/4406; A61K 45/06; A61P 35/00

(57) ABSTRACT

The present invention relates to a combination of an HDAC inhibitor and statins for use in the treatment of pancreatic cancer. Preferably the invention relates to a combination of valproic acid (VPA) or any of its salts and simvastatin (SIM). The combination of the invention synergistically improves the anti-proliferative and pro-apoptotic effect of conventional chemotherapy, as gemcitabine/taxol.

### FIELD OF THE INVENTION

The present invention relates to a combination of an HDAC inhibitor and statins for use in the treatment of pancreatic cancer. Preferably the invention relates to a combination of valproic acid (VPA) or any of its salts and simvastatin (SIM). The combination of the invention synergistically improves the anti-proliferative and pro-apoptotic effect of conventional chemotherapy, as gemcitabine/taxol.

### BACKGROUND

Despite all advances in cancer therapies, pancreatic ductal adenocarcinoma (PDAC) patients have very poor prognosis [Lambert et al. Semin Oncol, 2021], suggesting the urgent need of novel treatments for this disease. In this regard, repurposing non-oncology already-approved drugs, might be an attractive strategy to offer effective treatment options easily translatable in early clinical trials.

Valproic acid (VPA) is a generic low-cost anticonvulsant with histone deacetylase (HDAC) inhibitory activity, whose anticancer properties were demonstrated in tumor models including PDAC both in monotherapy [Luo D et al. Carcinogenesis, 2020] or combined with gemcitabine [Lin T et al. JECCR, 2019]. Authors of the present invention recently demonstrated the potential of HDAC inhibitors in sensitizing PDAC cells to gemcitabine/abraxane doublet [Roca M S et al. JECCR, 2022]. We are currently exploring VPA plus conventional chemotherapy in solid tumors clinical studies, overall confirming the feasibility and safety of this approach [Avallone A et al. BMC cancer, 2016; Budillon A et al. Ann Onc, 2018].

Statins, developed as lipid-lowering drugs by inhibiting HMG-COA reductase, have further demonstrated a direct antitumor effect in monotherapy or in combination with chemotherapy and target therapy in different tumor models including pancreatic cancer [Gupta V et al, Cancer Lett 2018].

Recently, the authors of the present invention demonstrated the preclinical synergistic antitumor interaction of VPA and the cholesterol lowering agent simvastatin in metastatic prostate cancer models, and the ability of the combined treatment to sensitize prostate cancer cells to docetaxel and to revert docetaxel resistance. Mechanistically, this effect has been related with the capacity of the combined approach to target the cancer stem cells compartment via the inhibition of the oncogene YAP [Iannelli F et al. JECCR, 2020].

The present invention reports for the first time data of a synergistic antitumor effect of VPA/SIM combination in pancreatic cancer models, demonstrating that the combined approach potentiates GEM/NP (gemcitabine/nab-paclitaxel) doublet treatment both in vitro and in vivo tumor models. Moreover, the invention provides evidences demonstrating that the mechanism of the synergistic antitumor interaction is at least in part dependent on the VPA/SIM-mediated reversion of TGF-β-regulated epithelial-to-mesenchymal (EMT) transition.

### SUMMARY OF THE INVENTION

The present invention relates to a combination comprising at least one HDAC inhibitor and at least one statin for use in the treatment of pancreatic cancer, alone or in combination with further at least one anti-cancer agent.

Preferably, the at least one HDAC inhibitor is selected from valproic acid or a salt thereof, panobinostat, vorinostat, entinostat or mocetinostat, and the statin is selected from simvastatin, atorvastatin, lovastatin; more preferably the at least one HDAC inhibitor is valproic acid and the statin is simvastatin.

Preferably, the at least one HDAC inhibitor is selected from valproic acid or a salt thereof, panobinostat, vorinostat, entinostat or mocetinostat, and the statin is selected from simvastatin, atorvastatin, lovastatin; more preferably the at least one HDAC inhibitor is valproic acid and the statin is simvastatin.

In a preferred embodiment the combination comprising at least one HDAC inhibitor and one statin is used in a subject that has responded to, or is resistant to, or has developed resistance to a first line therapy, preferably said the first line therapy comprises administration of gemcitabine and/or nab-paclitaxel.

In a preferred embodiment the combination comprising at least one HDAC inhibitor and one statin is used in the ratio of the at least one HDAC inhibitor and one statin of about 50:50 cytotoxic ratio. As used herein, the 50:50 cytotoxic ratio indicates that the two classes drugs are used in equipotent doses.

In a further preferred embodiment, the invention relates to a combination comprising at least one HDAC inhibitor and at least one statin for use in the treatment of pancreatic cancer, alone or in combination with further anti-cancer agent wherein the at least one HDAC inhibitor is selected from:
valproic acid at a concentration ranging from about 16 mm to about 0.5 mM; and/or
vorinostat at a concentration ranging from about 16 μM to about 0.125 μM; and/or
panobinostat at a concentration ranging from about 160 nM to about 2.5 nM; and/or
entinostat at a concentration ranging from about 16 μM to about 0.125 μM;
   and wherein the at least one statin is selected from:
simvastatin at a concentration ranging from about 8 μM to about 0.06 μM; and/or
atorvastatin at a concentration ranging from about 8 μM to about 0.06 μM; and/or
lovastatin at a concentration ranging from about 8 μM to about 0.06 μM.

In the clinical setting, both valproic acid and simvastatin are oral bioavailable drugs that were tested at dosages within the range of their non-cancer approved indications, being these dosages preclinically effective in combination treatment of the two drugs plus chemotherapy.

In a further embodiment the present invention relates to a combination consisting of one HDAC inhibitor and one statin for use in the treatment of pancreatic cancer, alone or in combination with further at least one anti-cancer agent, as indicated above; preferably the HDAC inhibitor is selected from valproic acid or a salt thereof, panobinostat, vorinostat, entinostat or mocetinostat, and the statin is selected from simvastatin, atorvastatin, lovastatin; more preferably the HDAC inhibitor is valproic acid and the statin is simvastatin.

In a further preferred embodiment, the combination of the invention is used with a further anti-cancer agent selected from one or more of a Btk tyrosine kinase inhibitor, an Erbb2 tyrosine kinase receptor inhibitor; an Erbb4 tyrosine kinase receptor inhibitor, an mTOR inhibitor, a thymidylate synthase inhibitor, an EGFR tyrosine kinase receptor inhibitor, an Epidermal growth factor antagonist, a Fyn tyrosine kinase inhibitor, a kit tyrosine kinase inhibitor, a Lyn tyrosine kinase inhibitor, a NK cell receptor modulator, a PDGF receptor antagonist, a PARP inhibitor, a poly ADP ribose polymerase inhibitor, a poly ADP ribose polymerase 1 inhibitor, a poly ADP ribose polymerase 2 inhibitor, a poly ADP ribose polymerase 3 inhibitor, a galactosyltransferase modulator, a dihydropyrimidine dehydrogenase inhibitor, an orotate phosphoribosyltransferase inhibitor, a telomerase modulator, a mucin 1 inhibitor, a mucin inhibitor, a secretin agonist, a TNF related apoptosis inducing ligand modulator, an IL17 gene stimulator, an interleukin 17E ligand, a Neurokinin receptor agonist, a cyclin G1 inhibitor, a checkpoint inhibitor, a PD-1 inhibitor, a PD-L1 inhibitor, a CTLA4 inhibitor, a topoisomerase I inhibitor, an Alk-5 protein kinase inhibitor, a connective tissue growth factor ligand inhibitor, a notch-2 receptor antagonist, a notch-3 receptor antagonist, a hyaluronidase stimulator, a MEK-1 protein kinase inhibitor; MEK-2 protein kinase inhibitor, a GM-CSF receptor modulator; TNF alpha ligand modulator, a mesothelin modulator, an asparaginase stimulator, a caspase-3 stimulator; caspase-9 stimulator, a PKN3 gene inhibitor, a hedgehog protein inhibitor; Smoothened receptor antagonist, an AKT1 gene inhibitor, a DHFR inhibitor, a thymidine kinase stimulator, a CD29 modulator, a fibronectin modulator, an interleukin-2 ligand, a serine protease inhibitor, a D40LG gene stimulator; TNFSF9 gene stimulator, a 2-oxoglutarate dehydrogenase inhibitor, a TGF-beta type II receptor antagonist, an Erbb3 tyrosine kinase receptor inhibitor, a cholecystokinin CCK2 receptor antagonist, a Wilms tumor protein modulator, a Ras GTPase modulator, an histone deacetylase inhibitor, a cyclin-dependent kinase 4 inhibitor A modulator, an estrogen receptor beta modulator, a 4-1BB inhibitor, a 4-1BBL inhibitor, a PD-L2 inhibitor, a B7-H3 inhibitor, a B7-H4 inhibitor, a BTLA inhibitor, a HVEM inhibitor, aTIM3 inhibitor, a GAL9 inhibitor, a LAG3 inhibitor, a VISTA inhibitor, a KIR inhibitor, a 2B4 inhibitor, a CD160 inhibitor and a CD66e modulator, or combination thereof.

Preferably said further anti-cancer agent is selected from bavituximab, IMM-101, CAP1-6D, Rexin-G, genistein, CVac, MM-D37K, PCI-27483, TG-01, LOAd-703, CPI-613, upamostat, CRS-207, NovaCaps, trametinib, Atu-027, sonidegib, GRASPA, trabedersen, nastorazepide, Vaccell, oregovomab, istiratumab, refametinib, regorafenib, lapatinib, selumetinib, rucaparib, pelareorep, tarextumab, PEGylated hyaluronidase, varlitinib, aglatimagene besadenovec, GBS-01, GI-4000, WF-10, galunisertib, afatinib, RX-0201, FG-3019, pertuzumab, DCVax-Direct, selinexor, glufosfamide, virulizin, yttrium (90Y) clivatuzumab tetraxetan, brivudine, nimotuzumab, algenpantucel-L, tegafur+gimeracil+oteracil potassium+calcium folinate, olaparib, ibrutinib, pirarubicin, Rh-Apo2L, tertomotide, tegafur+gimeracil+oteracil potassium, tegafur+gimeracil+oteracil potassium, masitinib, Rexin-G, mitomycin, erlotinib, adriamycin, dexamethasone, vincristine, cyclophosphamide, topotecan, taxol, interferons, platinum derivatives, taxane, paclitaxel, vinca alkaloids, vinblastine, anthracyclines, doxorubicin, epipodophyllotoxins, etoposide, cisplatin, rapamycin, methotrexate, actinomycin D, dolastatin 10, colchicine, emetine, trimetrexate, metoprine, cyclosporine, daunorubicin, teniposide, amphotericin, alkylating agents, chlorambucil, 5-fluorouracil, campthothecin, metronidazole, Gleevec, panitumumab, abarelix, aldesleukin, alemtuzumab, alitretinoin, allopurinol, altretamine, amifostine, anastrozole, arsenic trioxide, asparaginase, azacitidine, AZD9291, BCG Live, bevacuzimab, bexarotene, bleomycin, bortezomib, busulfan, calusterone, capecitabine, camptothecin, carboplatin, carmustine, celecoxib, cetuximab, chlorambucil, cladribine, clofarabine, cyclophosphamide, cytarabine, dactinomycin, darbepoetin alfa, daunorubicin, denileukin, dexrazoxane, docetaxel, doxorubicin (neutral), doxorubicin hydrochloride, dromostanolone propionate, epirubicin, epoetin alfa, estramustine, etoposide phosphate, etoposide, exemestane, filgrastim, floxuridine fludarabine, fulvestrant, gefitinib, gemcitabine, gemtuzumab, goserelin acetate, histrelin acetate, hydroxyurea, ibritumomab, idarubicin, ifosfamide, imatinib mesylate, interferon alfa-2a, interferon alfa-2b, irinotecan, lenalidomide, letrozole, leucovorin, leuprolide acetate, levamisole, lomustine, megestrol acetate, melphalan, mercaptopurine, 6-MP, mesna, methotrexate, methoxsalen, mitomycin C, mitotane, mitoxantrone, nandrolone, nelarabine, nofetumomab, oprelvekin, oxaliplatin, paclitaxel, palifermin, pamidronate, pegademase, pegaspargase, pegfilgrastim, pemetrexed disodium, pentostatin, pipobroman, plicamycin, porfimer sodium, procarbazine, quinacrine, rasburicase, rituximab, rociletinib, sargramostim, sorafenib, streptozocin, sunitinib maleate, talc, tamoxifen, temozolomide, teniposide, VM-26, testolactone, thioguanine, 6-TG, thiotepa, topotecan, toremifene, tositumomab, trastuzumab, tretinoin, ATRA, uracil mustard, valrubicin, vinblastine, vincristine, vinorelbine, zoledronate, zoledronic acid, pembrolizumab, nivolumab, IBI-308, mDX-400, BGB-108, MEDI-0680, SHR-1210, PF-06801591, PDR-001, GB-226, STI-1110, durvalumab, atezolizumab, avelumab, BMS-936559, ALN-PDL, TSR-042, KD-033, CA-170, STI-1014, FOLFIRINOX and KY-1003, and combination thereof

Even more preferably, the further anti-cancer agent is at least one of taxol, gemcitabine, nab-paclitaxel, cisplatin, capecitabine, irinotecan or combination thereof, more preferably is a combination of taxol and gemcitabine or a combination of nab-paclitaxel and gembcitabine or a combination of gemcitabine, nab-paclitaxel, cisplatin, capecitabine.

In a further preferred embodiment, the combination as above defined is used for the treatment of a pancreatic cancer selected from the group consisting of pancreatic adenocarcinoma, non-resectable pancreatic cancer, locally advanced pancreatic cancer, borderline resectable pancreatic cancer, locally advanced pancreatic ductal adenocarcinoma, borderline resectable pancreatic ductal adenocarcinoma, metastatic pancreatic cancer, chemotherapy-resistant pancreatic cancer, pancreatic ductal adenocarcinoma, squamous pancreatic cancer, pancreatic progenitor, immunogenic pancreatic cancer, aberrantly differentiated endocrine exocrine (ADEX) tumors, an exocrine pancreatic cancer, pancreatic intraepithelial neoplasia, intraductal papillary mucinous neoplasms, mucinous cystic neoplasms, mucinous pancreas cancer, adenosquamous carcinoma, signet ring cell carcinoma, hepatoid carcinoma, colloid carcinoma, undifferentiated carcinoma, undifferentiated carcinomas with osteoclast-like giant cells, a pancreatic cystic neoplasm, an islet cell tumor, a pancreas endrocrine tumor, or a pancreatic neuroendrocrine tumor.

The invention further relates to the combination comprising at least one HDAC inhibitor and one statin or to the combination consisting of one HDAC inhibitor and one statin, as defined above, wherein the at least one HDAC inhibitor and one statin are administered in a single dosage unit or separately, preferably said single dosage unit comprises at least one pharmaceutically acceptable excipient, preferably the single dosage unit or the separate dosage formulations are in the form of an oral, parenteral and/or topical dosage forms.

### BRIEF DESCRIPTION OF FIGURES

The invention will be now illustrated referring to the following figures.

FIG. 1. Pancreatic Cancer Cell lines features. A. Basal mRNA basal levels of EMT markers Vimentin and E-Cadherin in PDAC cell lines L3.6pl, BxPC3, COLO357, PANC28, PANC1, ASPC1 and MIAPACA2, in normal pancreatic cells HPDE cells, as well as in 2 established PDX-derived primary cells KPC ID11 and KPC ID95 were determined by qRT-PCR. B. Basal protein expression analysis shows EMT markers in PDAC cell lines L3.6pl, BxPC3, COLO357, PANC28, PANC1, ASPC1 and MIAPACA2, in normal pancreatic cells HPDE cells, as well as in 2 established cell line derived from C57/BL6 mice KPC ID11 and KPC ID95 were determined by western blot analysis. Cells were grown for 48 h before preparation of cell lysates. Proteins were separated by SDS-PAGE and transferred to PVDF membranes using standard protocols. The membranes were probed with specific antibodies and Ponceau Red as total protein loading controls. Data represents representative results of at least three independent experiments.

FIG. 2. Synergistic effect of Statins/HDACi Combination in PDAC cell lines. Synergistic effect of Statins/HDACi Combination on PDAC cell lines analyzed according to the T-C Chou and P. Talalay method. PANC28, PANC1, ASPC1, BxPC3, MIAPACA2, L3.6pl, KPC ID11 and KPC ID95 were plated as described (see Material and Methods) and treated for 96 h with single drugs or combination (50:50 ratio). Cells viability was evaluated using sulforhodamine B colorimetric assay and combination index (CI). The CI suggests the goodness of a drug combination and is mathematically calculated using Calcusyn Software (Biosoft, Cambridge, UK). In detail, a CI<0.8, CI<0.9, CI=0.9-1.1, and CI>1.1 indicated a strong synergism, synergism, additivity or antagonism, respectively (Terranova-Barberio M, J Exp Clin Cancer Res. 2017; 36(1):177). Data represent the combined results of three independent experiment (mean±DS); Abbreviations: VPA: Valproic Acid; SIM: Simvastatin; PAN: Panobinostat; VOR: Vorinostat; MS-275: Entinostat; ATOR: Atorvastatin; LOV: Lovastatin. Gray scale code was used to highlight antagonism, additivity, synergism and strong synergism.

FIG. 3. VPA plus SIMVASTATIN antiproliferative and pro-apoptotic effects in PDAC cell lines. A. Synergistic inhibition of colony formation by the VPA/SIM in ASPC1, PANC28, PANC1 and BxPC3 cell lines. Cells were plated at a very low concentration (50-200 cells/well) and after 24 h were treated at IC10, IC25 of VPA and SIM alone and in combination, the value are reported in the graph. Upper panel: representative imagines of one experiment; in the lower panel: the bar graphs represent the values of absorbance of colonies after SRB colorimetric assay. Statistical significance was determined by a 2 tailed, unpaired Student's T test (*P<0.05;** P<0.01). B.-C. Synergistic inhibition of microtissues formation by the VPA and SIM alone and in combination. 500 cancer cells (red ones-marked by cell tracker) and 1500 normal fibroblasts were plated in each well and after 24 h treated at the respective IC25. Representative images from Opera Phenix confocal microscopy (B). The graphics represent the number of viable cells in 3D cell culture based on quantitation of the ATP content. Results were obtained by a single experiment performed in triplicate (±S.D). Statistical significance was determined by a 2 tailed, unpaired Student's T test (*P<0.05;** P<0.01) (C). D. Western blot analysis of PARP1 cleavage expression, in PDAC cells untreated or treated with VPA and/or SIM at IC25 and IC50 for 24, 48 and 72 hours. β-actin expression serves as loading control.

FIG. 4. Apoptotic/necroptoic effect of VPA/SIM treatment in PC cell lines. Apoptosis evaluated by flow cytometry after Annexin V-FITC staining in PANC1, MDA-PANC28, ASPC1 and BXPC3 and cells, untreated or treated for 48 hours or 72 hours, with VPA and/or SIM at IC₂₅^{96h}, calculated as the drugs concentration that inhibits cell proliferation by 25% compared to the untreated control cells. In details, the cells were seeded in 96-well plate and 24 hours later VPA and SIM were administrated in escalation dosages starting respectively from 16 mM and 80 mM. After 96 hours the cells viability and IC₂₅ was evaluated using sulforhodamine B colorimetric assay.

FIG. 5. Combination of VPA/SIM, at low doses, plus gemcitabine/taxol induces synergistic cell growth inhibition and apoptosis in monolayer and spheroids culture in PDAC cells. A. Cells were treated for 96 h with increasing concentrations GEM/TAX alone or with fixed low doses of VPA/SIM (VPA 0.5 mM and SIM 0,625 μM). Antiproliferative effect was assessed in all PDAC cell lines by sulforhodamine B colorimetric assay (see Methods) and expressed as percentage of control. B Apoptosis was evaluated by Caspase 3/7 activity assay, in PANC1, PANC28 and ASPC1 cells untreated or treated for 24 h with VPA/SIM and/or GEM/TAX at the low doses (VPA 0.5 mM, SIM 0,625 μM, GEM 25 nM and TAX 1.25 nM) and evaluated by luminescence assay. C. Expression of cleaved PARP in PANC1, PANC28 and ASPC1 cell lines untreated or treated with VPA/SIM and/or GEM/TAX for 24 h was evaluated by western blotting. βActin was used as loading control. D-E Antitumor effect of VPA/SIM plus GEM/TAX in 3D spheroid system. Specifically, PANC1 (D) and ASPC1 (E) were seeded in sphere medium in low attachment support, to form 1st generation spheres. After 48 h spheres were disaggregated and plated in three different conditions: adherent condition and tested for cell viability (graph on the left), in sphere medium in low attachment support to form 2nd generation and tested for cell viability in 3D (graph in the middle) and in terms of induction of apoptosis (graph on the right). In detail, cells were seeded in sphere medium in low attachment support, to form 1st generation spheres for 48 h, then disaggregated and plated again in adherent condition and after 24 h from seeding cells were treated with VPA/SIM and/or GEM/TAX at the indicated doses for 96 h. Antiproliferative effect was assessed in all PDAC cell lines by sulforhodamine B colorimetric assay (Cell viability—SRB, graph on the left). Cells (40,000/mL) were seeded in sphere medium in low attachment support, to form 1st generation spheres for 48 h, then disaggregated and plated again in sphere medium in low attachment support to form 2nd generation spheres and concomitantly untreated or treated with VPA/SIM and/or GEM/TAX at the indicated doses for 72 h. Spheroids viability was assessed by luminescence assay (graph in the middle). For apoptosis induction, cells (40,000/mL) were seeded in sphere medium in low attachment support, to form 1st generation spheres for 48 h, then disaggregated and plated again in sphere medium in low attachment support to form 2nd generation spheres and concomitantly untreated or treated with VPA/SIM and/or GEM/TAX at the indicated doses for 24 h. Apoptosis was evaluated by Caspase 3/7 activity assay. F. Synergistic inhibition of colony formation induced by the VPA and SIM in combination with chemotherapy in PANC1, ASPC1, PANC28, and BxPC3 cell lines. Cells were plated at a very low concentration and after 24 h were treated at IC10 of VPA and SIM alone and/or in combination with chemotherapy. Representative imagines of one experiment

FIG. 6. VPA/SIM synergistically with gemcitabine/taxol reduces PANC1 cell migration capability by targeting TGFβ-induced EMT. A-B VPA/SIM in combination with GEM/TAX reduces vimentin and induces E-cadherin expression evaluated by fluorescence confocal microscopy in PANC1 cells untreated or treated as indicated for 48 h. Cells were stained with Vimentin antibody (secondary antibody Alex Fluor488) and E-cadherin antibody (secondary antibody Alex Fluor488) and DAPI for nuclei detection (blue). Quantitative measurements were made by Harmony software (PerkinElmer). C. EMT markers Vimentin and E-cadherin and pro-apoptotic markers, cleaved PARP and cleaved Caspase3, in Panc1 cell lines untreated or treated with VPA and/or SIM and/or GEM/TAX for 48 h was evaluated by western blotting. CDK4 and βActin were used as loading control. D. Vimentin, ZEB1 and CDH1 mRNA expression evaluated by RT-PCR in PANC1 cells untreated or treated with VPA or SIM at the low doses (0.5 mM and 0,625 μM respectively) for 48 h; the values represent the means±S.D. of technical triplicates. E. The network was generated by Ingenuity Pathway Analysis (IPA) using “Vimentin (VIM), E-Cadherin (CDH1), HMGCR and HDACs” search. Network genes are visualized by proper symbols, which specify the functional nature of the correspondent protein. Each node represents a gene and its direct (represented by solid lines) and indirect (represented by dotted lines) association with other genes. Genes used as input are highlighted dark gray, whereas TGFβ, in light gray, came out as hub. F. Relative mRNA expression of Vimentin, ZEB1 and CDH1 expressed in PANC1 cells measured by real-time RT-PCR, untreated or treated with VPA/SIM at the low doses (0.5 mM and 0.625 μM respectively), with and without TGFβ (5 ng/ml) stimulation for 8 h. G. TGFβ mRNA expression evaluated by RT-PCR in PANC1 cells untreated or treated for the indicated time points with VPA or SIM at the low doses (0.5 mM and 0.625 μM respectively); the values represent the means±S.D. of technical triplicates.

FIG. 7. VPA/SIM synergistically with gemcitabine/taxol reduces PANC1 cell migration and invasion capability and impacts on PDAC microenvironment. A. Migration assay was performed to evaluated by wound-healing assay. PDAC cells were seeded to 90% of confluence in 96 well cell carrier ultra (PerkinElmer). A sterile 10-μl pipette tip, was used 24 hours after plating, to longitudinally scratch a constant-diameter stripe in the confluent monolayer to simulate a wound. Then the cells were untreated or exposed to VPA, SIM and GEM/TAX until the wound resulted almost completely closed. At the indicated time wells were photographed by Opera Phenix microscope (PerkinElmer) air objective magnification 20×. Quantitative measurements were made by determining the distances between the wound-edges in by Harmony software (PerkinElmer). B. Invasion assay was performed in transwell, using 8 μm pore size PVPF filters and expressed as % of invading cells, upon 48 h exposure to VPA, SIM alone and in combination with GEM/TAX. C. HPaSteC cells were untreated or treated with VPA and/or SIM for 24 h. Cells were collected and stained with GFAP-APC for 30 min at 4° C. for evaluation of activated stellate cells and assayed by flow cytometry. D. HPaSteC were treated for 96 h with increasing concentrations of VPA and SIM alone and in combination. Antiproliferative effect was assessed by sulforhodamine B colorimetric assay (see Methods) and expressed as percentage of control. E. HPaSteC cells were treated with conditioned medium from PANC1 previously untreated or treated with VPA and/or SIM for 24 h. HPaSteC were exposed to PANC1 conditioning for 24 h and then collected and stained with GFAP-APC for 30 min at 4° C. for evaluation of activated stellate cells and assayed by flow cytometry. F. HPaSteC cells were treated with conditioned medium from PANC1 transfected with empty vector (PANC1_EV), wild-type YAP (YAPwt)-transfected and YAP5SA (constitutively active)-transfected untreated or treated with VPA and/or SIM for 24 h. HPaSteC were exposed to PANC1 conditioning for 24 h and then collected and stained with GFAP-APC for 30 min at 4° C. for evaluation of activated stellate cells and assayed by flow cytometry. G. Western blotting analysis of YAP subcellular localization upon VPA and/or SIM treatment in PANC1 cells. γTubulin and PARP were used as loading control. H. Microtissues obtained by mixing PDAC cells marked using a green fluorescent probe-cell traker with human pancreatic stellate cells with red probe (PKH-26 Sigma Aldrich) were cultured in the ULA System (PerkinElmer). The 3D microtissue model was obtained using normal fibroblasts as scaffold for the PDAC cell lines in a ratio of 3:1 and untreated or treated with drugs for 96 h. 3D microtissues were maintained in the incubator and photographed by Opera Phenix microscope (PerkinElmer) air objective magnification 5× and mean volume was scored by Harmony software.

FIG. 8. In vivo synergistic antitumor effect of valproic acid and simvastatin in combination with gemcitabine/Nab-paclitaxel in Panc1-LUC orthotropic model. A. Schematic view of the in vivo experiment: VPA/SIM plus Gem/NP effects on PDAC orthotopic xenograft tumor growth. LUC-transfected PANC1 cells (0.5×106 cells in 50 μL of PBS) were injected directly into the pancreas of athymic mice as described in the Materials and Methods. One week after injection, mice were treated with VPA/SIM and/or GEM/NP, the four drugs in combination, or their vehicles. B. Digital grayscale image, for the five mice/groups, at day 5, day 12 and day 19 (follow up) overlaid to a pseudo-color image representing the spatial distribution of detected photons emerging from active luciferase. C Tumor volume was quantified as the sum of all detected photons within the region of the tumor/s. The measurement for each single mouse at the indicated time point was compared with the respective values at day 1 (T0). Relative fold increase values were reported in the graph. D Tumor volume was quantified as the sum of all detected photons within the region of the tumor/s as a mean of each measurement for the four groups at the indicated time point. E Body weight measured twice a week. F. Serum level of TGF-β from control and treated mice after 2 weeks of treatment, evaluated by Quantikine ELISA TGFβ1 immunoassay (see material and methods) Q. Results are expressed as mean±SD from 3 mice. G. Representative sections of IHC staining for Masson's trichrome of orthotopic pancreatic tumor sections. Scale bar 50 μm, magnitude 20×. Collagen fibers are stained blue and nuclei are black. The image on the left shown a clearly delineated blue indicating the presence of fibrotic areas within the tumor quantified as ≥10% of fibrosis. Whereas image on the right shown Masson's trichrome staining a presence of collagen fibers in blue <of 10% indicating a reduced fibrous tissue. The table shown the number of mice/group characterized by the presence of fibrosis ≥10% or <of 10%.

FIG. 9. In vivo synergistic antitumor effect of valproic acid and simvastatin in combination with gemcitabine/Nab-paclitaxel in Panc1 heterotopic model. A. Schematic view of the in vivo experiment: VPA/SIM plus Gem/NP effects on PDAC etherotopic xenograft tumor growth. B. Relative tumor volume curves for PANC1 xenografts. Means±SD tumour volume measured at pre-specified time points. C. Serum level of TGF-β from control and treated mice after 2 weeks of treatment, evaluated by Quantikine ELISA TGFβ1 immunoassay (see material and methods) Q. Results are expressed as mean±SD from 3 mice. D-E-F. GOT, GPT, and creatinine serum levels in mice sacrificed at the end of treatment.

### DETAILED DESCRIPTION OF THE INVENTION

Within the present invention it was surprisingly found that a combination comprising at least one HDAC inhibitor and one statin, preferably the VPA/SIM combination, both in vitro and in vivo, used at low dosages, synergistically improves the anti-proliferative and pro-apoptotic effect of gemcitabine/taxol conventional chemotherapy. Said combination of HDAC inhibitor(s) and statin(s) has anticancer activity and is for use in the treatment of pancreatic cancer.

Mechanistically, VPA/SIM treatment, alone or in combination with chemotherapy, induced e-Cadherin and impaired vimentin and ZEB-1 expression, functionally linked to the synergistic inhibition of cell migration and invasion.

Ingenuity Pathway Analysis (IPA) highlighted a protein network connecting HDACs and HMGCR, the targets of VPA and SIM respectively, with the two main EMT markers. Here, TGFβ emerged as a hierarchical dominant network-node.

VPA/SIM inhibited TGFβ transcription and TGFβ-regulated EMT gene expression in PDAC cells. Moreover VPA/SIM treatment impaired YAP nuclear translocation, in line with the data obtained in prostate cancer models [Iannelli F et al. JECCR, 2020].

VPA/SIM combination affected the activation of human pancreatic stellate cells (HPaSteC) as shown by impairment of glial fibrillary acidic protein (GFAP) expression, without affecting their proliferation. This effect was induced both directly by VPA/SIM treatment and upon treatment of HpaSteC with conditioned media from a PDAC cell line, PANC1, untreated or treated with VPA/SIM. Moreover, the latest inhibition was reverted by the use of same PDAC cell line transfected with constitutive active YAP oncogene (YAP5SA), confirming its involvement in the crosstalk between PDAC and HpaStec cells

Overall, the present invention provides a novel combination strategy, based on the combination of an HDAC inhibitor and a statin, in particular a combination comprising two safe and generic drugs, able to sensitize a widely employed regimen in metastatic PDAC patients. On this basis, the inventors designed a randomized phase II clinical study of VPA combined with simvastatin and gemcitabine/nab-paclitaxel or gemcitabine/nab-paclitaxel/cisplatin/capecitabine based regimens in untreated metastatic pancreatic adenocarcinoma patients that will start enrollment shortly (VESPA trial—EudraCT n. 2022-004154-63).

In the clinical setting, both valproic acid and simvastatin are oral bioavailable drugs that were tested at dosages within the range of their non-cancer approved indications, being these dosages preclinically effective in combination treatment of the two drugs plus chemotherapy.

The invention also relates to a method for treating pancreatic cancer comprising the administration of at least one HDAC inhibitor and one statin in a subject in need thereof. Preferably said method comprises administering at least one HDAC inhibitor selected from valproic acid or a salt thereof, panobinostat, vorinostat, entinostat or mocetinostat, and at least one statin selected from simvastatin, atorvastatin, lovastatin.

Unless otherwise defined herein, scientific and technical terms used in connection with the present disclosure shall have the meanings that are commonly understood by those of ordinary skill in the art. Further, unless otherwise required by context, singular terms shall include the plural and plural terms shall include the singular as is considered appropriate to the context and/or application. The various singular/plural permutations may be expressly set forth herein for the sake of clarity.

As used herein, the term “combination” refers to a single composition/formulation comprising an HDAC inhibitor and a statin or a respective pharmaceutically acceptable salt or derivative thereof; or a kit comprising the HDAC inhibitor and the statin or a respective pharmaceutically acceptable salt or derivative thereof as separate formulations; or separate formulations/dosage forms not in the form of a kit as long as the effect achieved is commensurate with the intended purpose of the invention, i.e., to work for treatment of pancreatic cancer. Said combination comprises one or more pharmaceutically acceptable excipients. Accordingly, the separate formulations comprising the HDAC inhibitor and the statin or a respective pharmaceutically acceptable salt or derivative thereof may be administered simultaneously, or one after the other in any order. Preferably the HDAC inhibitor is valproic acid or a salt thereof, panobinostat, vorinostat, entinostat or mocetinostat, and the statin is selected from simvastatin, atorvastatin, lovastatin; more preferably the HDAC inhibitor is valproic acid and the statin is simvastatin.

The use of the expression “at least” or “at least one” suggests the use of one or more elements or ingredients or quantities, as the use may be in the embodiment of the disclosure to achieve one or more of the desired objects or results. Throughout this specification, the word “comprise”, or variations such as “comprises” or “comprising” or “containing” or “has” or “having” wherever used, will be understood to imply the inclusion of a stated element, integer or step, or group of elements, integers or steps, but not the exclusion of any other element, integer or step, or group of elements, integers or steps. Reference throughout this specification to “one embodiment” or “an embodiment” means that a particular feature, structure or characteristic described in connection with the embodiment may be included in at least one embodiment of the present disclosure. Thus, the appearances of the phrases “in one embodiment” or “in an embodiment” in various places throughout this specification may not necessarily all refer to the same embodiment. It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. The term “about” as used herein encompasses variations of +/−10% and more preferably +/−5%, as such variations are appropriate for practicing the present invention.

The term “histone deacetylase” or “HDAC”, as used herein, refers to an enzyme that removes acetyl groups from histones. There are currently 18 known HDACs, which are classified into four groups. Class I HDACs, includes HDAC1-3 and HDAC8. Class II HDACs include HDAC4-7 and HDAC9-10. Class III HDACs (also known as the sirtuins) include SIRT1-7. Class IV HDACs, which contains only HDAC11, has features of both Class I and II HDACs. The term “histone deacetylase inhibitor” or “HDAC inhibitor” or “HDACi” as used herein, refers to a compound natural or synthetic that inhibits histone deacetylase activity. There exist different classes of HDACi in function of their selectivity for their substrates divided in classical HDACi, selective class I HDACi and selective class II HDACi. A “classical HDACi” (also known as pan-HDACi) refers thus to a compound natural or not which has the capability to inhibit the histone deacetylase activity independently of the class of HDACs. Therefore a classical HDACi is a non selective HDACi. By “non selective” it is meant that said compound inhibits the activity of classical HDACs (i.e. class I, II and IV) with a similar efficiency independently of the class of HD AC. Examples of classical HDACi include, but are not limited to, Belinostat (PDX-101), Vorinostat (SAHA) and Panobinostat (LBH-589). A “selective class I HDACi” is selective for class HDACs (i.e. HD AC 1-3 and 8) as compared with class II HDACs (i.e. HDAC4-7, 9 and 10). By “selective” it is meant that selective class I HDACi inhibits class I HDACs at least 5-fold, preferably 10-fold, more preferably 25-fold, still preferably 100-fold higher than class II HDACs. Selectivity of HDACi for class I or class II HDACs may be determined according to previously described method (Kahn et al. 2008). Examples of selective class I HDACi include, but are not limited to, valproic acid (VPA), Romidepsin (FK-228) and Entinostat (MS-275). A “selective class II HDACi” is selective for class II HDACs (i.e. HDAC4-7, 9 and 10) as compared with class I HDACs (i.e. HD AC 1-3 and 8). By “selective” it is meant that selective class II HDACi inhibits class II HDACs at least 5-fold, preferably 10-fold, more preferably 25-fold, still preferably 100-fold higher than class I HDACs. Examples of selective class II HDACi include, but are not limited to, tubacin and MC-1568 (aryloxopropenyl) pyrrolyl hydroxamate). HDAC inhibition relies mainly on a mechanism based on the inhibition of the HDAC enzymatic activity which can be determined by a variety of methods well known by the skilled person. Usually, these methods comprise assessing the lysine deacetylase activity of HDAC enzymes using colorimetric HDAC assays. Commercial kits for such techniques are available (see for example, Histone Deacetylase (HDAC) Activity Assay Kit (Fluorometric) purchased from Abeam or Sigma-Aldrich). These methods are ideal for the determination of IC50 values of known or suspected HDAC inhibitors. Many HDAC inhibitors are known and, thus, can be synthesized by known methods from starting materials that are known, may be available commercially, or may be prepared by methods used to prepare corresponding compounds in the literature. A preferred class of HDAC inhibitors are hydroxamic acid inhibitors which are disclosed e. g. in WO 97/35990, US-A 5, 369, 108, US-A 5, 608, 108, US-A 5, 700, 811, WO 01/18171, WO 98/55449, WO 93/12075, WO 01/49290, WO 02/26696, WO 02/26703, JP 10182583, WO 99/12884, WO 01/38322, WO 01/70675, WO 02/46144, WO 02/22577 and WO 02/30879. Other HDAC inhibitors which can be included within the compositions of the present invention are cyclic peptide inhibitors, and here it can be referred e. g. to U.S. Pat. No. 5,620,953, US-A 5, 922, 837, WO 01/07042, WO 00/08048, WO 00/21979, WO 99/11659, WO 00/52033 and WO 02/0603. Suitable HDAC inhibitors are also those which are based on a benzamide structure which are disclosed e. g. in Proc. Natl. Acad. Sci. USA (1999), 96:4592-4597, but also in EP-A 847 992, U.S. Pat. No. 6,174,905, JP 11269140, JP 11335375, JP 11269146, EP 974 576, WO 01/38322, WO 01/70675 and WO 01/34131. The HDAC inhibitors may be used under any pharmaceutically acceptable form, including without limitation, their free form and their pharmaceutically acceptable salts or solvates. The term “pharmaceutically acceptable salts” refers to salts prepared from pharmaceutically acceptable, preferably non-toxic, bases or acids including mineral or organic acids or organic or inorganic bases. Such salts are also known as acid addition and base addition salts. Examples of pharmaceutically acceptable salts are discussed in Berge et al., 1977, “Pharmaceutically Acceptable Salts,” J. Pharm. Sci., Vol. 66, pp. 1-19. More particularly, examples of suitable HDAC inhibitors according to the invention include, but are not limited to the compounds listed in Table 1 below.

TABLE 1
Examples of HDAC inhibitors useful within the context
of the invention

| Class | Agent |
|---|---|
| Hydroxamates | Belinostat (PDX-101) |
| Hydroxamates | Vorinostat (SAHA) |
| Hydroxamates | Panobinostat (LBH-589) |
| Hydroxamates | Givinostat (IFT2357) |
| Hydroxamates | Abexinostat (PCI-24781) |
| Hydroxamates | Trichostatin A (TSA) |
| Hydroxamates | Quisinostat (JNJ-26481585) |
| Hydroxamates | Pracinostat (SB939) |
| Hydroxamates | Resminostat (4SC-201) |
| Hydroxamates | Tefinostat (CHR-2845) |
| Hydroxamates | Dacinistat (LAQ-824) |
| Hydroxamates | CG20075 |
| Hydroxamates | Citarinostat (ACY-241) |
| Hydroxamates | Ricolinostat (ACY-1215) |
| Hydroxamates | MC-1568 |
| Benzamides | Mocetinostat (MGCD0103) |
| Benzamides | Entinostat (MS-275) |
| Benzamides | Tacedinaline (CI994) |
| Benzamides | Chidamide (CS055/HBI-8000) |
| Benzamides | BML-210 |
| Cyclic peptides | Romidepsin (FK-228, Depsipeptide) |
| Cyclic peptides | Trapoxin A |
| Cyclic peptides | Apicidine |
| Short-chain fatty acid (SCFA) | Sodium Phenylbutyrate |
| Short-chain fatty acid (SCFA) | Valproic acid (VPA) |
| Short-chain fatty acid (SCFA) | Sodium butyrate (S4125) |

In a preferred embodiment, the HDAC inhibitor is selected from the group consisting of valproic acid, belinostat (PXD-101), vorinostat (SAHA), entinostat (MS-275) panobinostat (LBH-589), mocetinostat (MGCD0103), chidamide (HBI-8000) romidepsin (FK-228) and Trichostatin A (TSA).

Valproic acid has the chemical name of 2-propylpentanoic acid. Divalproex sodium is the stable, coordinated compound of sodium valproate and valproic acid. As used herein, the term valproic acid includes valproic acid itself, salts of valproic acid such as sodium valproate, divalproex sodium and other derivatives valproic acid.

Belinostat (also known as PXD-101) has the chemical name (2E)-N-hydroxy-3-[3-(phenylsulfamoyl)phenyl]prop-2-enamide. Vorinostat is currently commercially available for oral administration in the U.S. under the brand name Zolinza® (Merck Sharp & Dohme Corp). Panabinostat (also known LBH-589) has the chemical name 2-(E)-N-hydroxy-3-[4 [[[2-(2-methyl-1H-indol-3-yl)ethyl]amino]methyl]phenyl]-2-propenamide. Panabinostat lactate is currently commercially available for oral administration in the U.S. under the brand name Farydak® (Novartis). Mocetinostat (also known as MGCD0103) has the chemical name N-(2-Aminophenyl)-4-[[(4-pyridin-3-ylpyrimidin-2-yl)amino]methyl]benzamide. Chidamide (also known as HBI-8000) has the chemical name N-(2-Amino-5-fluorophenyl)-4-[[[1-oxo-3-(3-pyridinyl)-2-propen-1-yl]amino]methyl]-benzamide. Entinostat (also known as MS-275) has the chemical name N-(2-aminophenyl)-4-N-(pyridine-3-yl) methoxycarbonylamino-methyl]-benzamide. Romidepsin is a natural product which was isolated from Chromobacterium violaceum by Fujisawa Pharmaceuticals. Romidepsin (also known as FK-228) is a bicyclic depsipeptide [1S,4S,7Z,10S,16E,21R)-7-ethylidene-4,21-bis(lmethylethyl)-2-oxa-12,13-dithia-5,8,20,23-tetraazabicyclo[8.7.6]tricos-16ene-3,6,9,19,22-pentone]. Trichostatin-A (TSA) (also known as TSA) hs the chemical name of 2,4-Heptadienamide, 7-[4-(dimethylamino)phenyl]-N-hydroxy-4,6-dimethyl-7-oxo-, (2E,4E,6R). TSA is an organic compound that serves as an antifungal antibiotic and selectively inhibits the class I and II mammalian histone deacetylase (HDAC) families of enzymes, but not class III HDACs (i.e., sirtuins). It is a member of a larger class of histone deacetylase inhibitors (HDIs or HDACIs) that have a broad spectrum of epigenetic activities. Thus, TSA has some potential as an anti-cancer drug (Drummond D C, et al. (2005) Annu Rev Pharmacol Toxicol. 45:495-528.)

As used herein, the term “treatment” means reversing, alleviating, inhibiting the progress of, or preventing the disorder or condition to which such term applies, or reversing, alleviating, inhibiting the progress of, or preventing one or more symptoms of the disorder or condition to which such term applies. By a “therapeutically effective amount” is meant a sufficient amount to be effective, at a reasonable benefit/risk ratio applicable to any medical treatment. It will be understood, however, that the total daily usage will be decided by the attending physician within the scope of sound medical judgment. The specific therapeutically effective dose level for any particular patient in need thereof will depend upon a variety of factors including the age, body weight, general health, sex and diet of the patient, the time of administration, route of administration, the duration of the treatment; drugs used in combination or coincidental with the and like factors well known in the medical arts. For example, it is well known within the skill of the art to start doses of the compound at levels lower than those required to achieve the desired therapeutic effect and to gradually increase the dosage until the desired effect is achieved.

Statins are a class of drugs that are widely prescribed in the management and prevention of cardiovascular disease. Studies have suggested that statins can lower low-density lipoprotein (LDL) cholesterol levels by up to 551 and cardiovascular events by 20-301 (Postmus, 2014}. Statins are 3˜hydroxy-3-raethylglutaryl-coenzyme A (HMG CoA) reductase inhibitors. HMG CoA reductase is the rate-limiting enzyme in cholesterol synthesis. By competitively inhibiting HMG CoA reductase activity, statins decrease cellular cholesterol concentration, which activates a cellular signaling cascade culminating in the activation of sterol regulatory element binding protein (SREBP). SREBP is a transcription factor that up-regulates expression of the gene encoding the LDL receptor. LDL receptors are responsible for receptor-mediated endocytosis of LDL cholesterol. Thus, increased LDL receptor expression causes increased uptake of plasma LDL and consequently decrease plasma LDL-cholesterol concentration. The best-selling statin drug is atorvastatin, marketed as LIPITOR and manufactured by Pfizer. Lipitor is available in tablet form for daily oral administration, each tablet containing 10, 20, 40, or 80 mg atorvastatin. In addition to Lipitor, statins are also commercially available as single-ingredient products as Lescol (fluvastatin), Mevacor (lovastatin), Altoprev® (lovastatin extended-release), Livalo® (pitavastatin), Pravachol (pravastatin), Crestor® (rosuvastatin), and Zocor® (simvastatin). Statins are also commercially available as combination products as Advicor (lovastatin/niacin extended-release), Simcor® (simvastatin/niacin extended-release), and Vytorin (simvastatin/ezetimibe).

The statins employed in the combination therapy of the present disclosure are selected from a group comprising simvastatin, atorvastatin, lovastatin, rosuvastatin, fluvastatin, pitavastatin, pravastatin, or any combination thereof, preferably simvastatin, atorvastatin, lovastatin, more preferably simvastatin. Any of these statins are expected to work in the present combinations in view of a study conducted by Jing et al. where they investigated the anticancer effects of different statins and observed that almost all statins exhibited anticancer activity (“In vitro and in vivo anticancer effects of mevalonate pathway modulation on human cancer cells”, Br J Cancer. 2014 Oct. 14; 111(8):1562-71)

In some embodiments, the therapeutically effective amount of the HDAC inhibitor or of valproic acid or a pharmaceutically acceptable salt or derivative thereof is ranging from about 1 mg to about 2500 mg per day, preferably about 100 mg to about 2500 mg, about 200 mg to about 2500 mg, about 300 mg to about 2500 mg, about 400 mg to about 2500 mg, about 500 mg to about 2500 mg, about 600 mg to about 2500 mg, about 700 mg to about 2500 mg, about 800 mg to about 2500 mg, about 900 mg to about 2500 mg, about 1000 mg to about 2500 mg, about 1100 mg to about 2500 mg, about 1200 mg to about 2500 mg, about 1300 mg to about 2500 mg, about 1400 mg to about 2500 mg, about 1500 mg to about 2500 mg, about 1600 mg to about 2500 mg, about 1700 mg to about 2500 mg, about 1800 mg to about 2500 mg, about 1900 mg to about 2500 mg, about 2000 mg to about 2500 mg, about 2100 mg to about 2500 mg, about 2200 mg to about 2500 mg, about 2300 mg to about 2500 mg, or about 2400 mg to about 2500 including and mg per day, values ranges therebetween.

In some embodiments, the therapeutically effective amount of the statin or a pharmaceutically acceptable salt or derivative thereof is from about 1 mg to about 200 mg per day, preferably 1 mg to about 100 mg or about 50 mg to about 100 mg per day, including values and ranges there between.

In some embodiments, the foregoing values and ranges are merely suggestive. Dosages are altered depending on a number of variables, including, for example, the activity of the compound used, the disease, disorder or condition to be treated, the mode of administration, the requirements of the individual subject, the severity of the disease, disorder or condition being treated, and the judgment of the practitioner. A dose is modulated to achieve a desired pharmacokinetic or pharmacodynamics profile, such as a desired or effective blood profile.

The combination of the invention also comprises a further anti-cancer agent. The further anticancer agent can be a taxane. The term “taxane” may refer to any chemical analogue which exerts its anticancer effect by stabilization of the tubulin microtubules involved in cell division. Examples of taxanes that may be combined the combination of the HDAC inhibitor and statin according to the present inventions include: (2aR,3aR,4aR,6R,9S,11S,12S,12aR,12bS)-6,12b-diacetoxy-9-[3(S)-(tert-butoxycarbonylamino)-2(R)-hydroxy-3-phenylpropionyloxy]-12-benzoyloxy-11-hydroxy-8,13,13-trimethyl-2a,3,3a,4,5,6,9,10,11,12,12a, 12b-dodecahydro-1H-7,11-methanocyclodeca[3,4]-cyclopropa[4,5]benz[1,2-b]oxet-5-one dihydrate; paclitaxel (Taxol), BMS-184476 (7-methylthiomethylpaclitaxel); BMS-188797; BMS-275183; BMS-188797; BMS-109881; CYC-3204 (a penetratin-paclitaxel conjugate); Taxoprexin; DJ-927; Docetaxel (Taxotere™); Larotexel (XRP9881; RPR-109881A); XRP6258 (RPR112658); Milataxel (MAC-321); MST 997; MBT-206; NBT-287; Ortataxel; Protax-3; PG-TXL; PNU-166945; PNU-106258; Orataxel (BAY 59-8862; IDN 5109; semisynthetic taxane); TPI-287; Protaxel and MAC-321 (Taxalog). Examples of formulations for taxanes include: conventional formulations of paclitaxel or docetaxel, for example the currently approved Taxol™ and Taxotere™ formulations; formulations with biocompatible polymers, particularly proteins such as albumin, more particularly nano-particle or micro-particle formulations of paclitaxel or docetaxel with albumin, for example Abraxane™ or nab-paclitaxel (described in U.S. Pat. Nos. 5,439,686 and 6,749,868) or NAB-docetaxel (described in, for example U.S. 20080161382, US20070117744 and US20070082838); polymer conjugates, particularly polymer conjugates of paclitaxel or docetaxel, more particularly conjugates of docetaxel or paclitaxel with poly-L-glutamate, for example Opaxio (also known as Xyotax, paclitaxel poliglumex, CT-2103 and described in for example Li C.; Poly (L-glutamic acid)—anticancer drug conjugates; Adv. Drug Deliv. Rev. 2002; 54; 695-713); conjugates of docetaxel or paclitaxel with a fatty acid, particularly conjugates of paclitaxel or docetaxel with docosahexaenoic acid (DHA), for example, Taxoprexin (DHA-paclitaxel, described in for example Bradley M O et al. Tumor targeting by covalent conjugation of a natural fatty acid to paclitaxel; Clin. Cancer Res. 2001; 7:3229-38); microparticle compositions such as the porous microparticle formulations described in U.S. Pat. No. 6,645,528, for example the microparticle formulation of paclitaxel AI-850, comprising paclitaxel nanoparticles in a porous, hydrophilic matrix, composed primarily of a sugar; and emulsions of paclitaxel or docetaxel in vitamin E, for example Tocosol.

The further anti-cancer agent can also be gemcitabine, a broad-spectrum antimetabolite and deoxycytidine analogue with antineoplastic activity, and/or cisplatin (cis-diamine, dichloroplatinum (II), CAS No. 15663-27-1) and/or capecitabine (XELODA®, Roche). As used herein, “pancreatic cancer” includes or refers collectively to the different types of pancreatic cancers including pancreatic adenocarcinoma, non-resectable pancreatic cancer, locally advanced pancreatic cancer, borderline resectable pancreatic cancer, locally advanced pancreatic ductal adenocarcinoma, borderline resectable pancreatic ductal adenocarcinoma, metastatic pancreatic cancer, chemotherapy-resistant pancreatic cancer, pancreatic ductal adenocarcinoma, squamous pancreatic cancer, pancreatic progenitor, immunogenic pancreatic cancer, aberrantly differentiated endocrine exocrine (ADEX) tumors, an exocrine pancreatic cancer, pancreatic intraepithelial neoplasia, intraductal papillary mucinous neoplasms, mucinous cystic neoplasms, mucinous pancreas cancer, adenosquamous carcinoma, signet ring cell carcinoma, hepatoid carcinoma, colloid carcinoma, undifferentiated carcinoma, undifferentiated carcinomas with osteoclast-like giant cells, a pancreatic cystic neoplasm, an islet cell tumor, a pancreas endrocrine tumor, or a pancreatic neuroendrocrine tumor.

The combination of the invention may be formulated in a dosage form including a pharmaceutically acceptable excipient or carrier. The pharmaceutically acceptable excipient or carrier may include but is not limited to at least one of ion exchangers, alumina, aluminum stearate, lecithin, serum proteins, human serum albumin, buffer substances, phosphates, glycine, sorbic acid, potassium sorbate, partial glyceride mixtures of saturated vegetable fatty acids, water, salts, electrolytes, protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, waxes, polyethylene glycol, starch, lactose, dicalcium phosphate, microcrystalline cellulose, sucrose, dextrose, talc, magnesium carbonate, kaolin; non-ionic surfactants, edible oils, physiological saline, bacteriostatic water, Cremophor EL™ (BASF, Parsippany, N.J.), and phosphate buffered saline (PBS).

### Materials and Methods

### Cell Lines

The Human pancreatic cancer cell lines PANC1, ASPC1, BxPC3, L3.6pl, COLO357, MIAPACA2 and the hTERT immortalized foreskin fibroblast BJhTERT were purchased from the American Type Culture Collection (ATCC, Rockville, MD, USA). PANC28 cell line was obtained from the laboratory of Dr Marsha L. Fraizer and Dr Douglas B Evans (Frazier, M. L., et al. International Journal of Pancreatology 19, 31-38-1996). The established PDX-derived primary cells KPC ID11 and KPC ID95 were kindly given by Bruno Sainz Lab in Madrid Alonso-Nocelo M, Sainz B et al. Gut. 2023 February; 72(2):345-359). The Stellate cells (HPastec) were purchased from the ScienCell research laboratories (Carlsbad, CA, USA) (Robinson, B. K., et al . . . (2016) Biology Open. VOL 5). In adherent condition PANC1, ASPC1, L3.6pl, COLO357, MIAPACA2, BJhTERT, PANC28, KPC ID 11 and ID95 cell lines were maintained as monolayer cultures and cultured in Dulbecco's modified Eagle's medium (DMEM) containing 4.5 g/L glucose, glutamine, and nonessential amino acids and supplemented with 10% heat-inactivated fetal bovine serum and penicillin (100 IU/mL)-streptomycin (100 μg/mL). BxPC3 cell line were grown in RPMI (Roswell Park Memorial Institute) supplemented with 10% fetal bovine serum (FBS, Cambrex, Belgium) heat-inactivated, 50 units/ml penicillin (Cambrex, Belgium), 500 g/ml streptomycin (Cambrex, Belgium), and glutamine 4 mM. HPaStec were cultured in stellate cell medium (SteCM). Cultures were maintained in a humidified atmosphere of 95% air and 5% CO2 at 37° C. All cell lines were regularly inspected for mycoplasma. The cells have been authenticated with short tandem repeat profile generated by LGC Standards. PANC1_LUC cell line stably transduced with RediFect firefly luciferase lentiviral particles (Catalog #CLS960004, PerkinHelmer) was obtained by lentiviral infection and selected using puromycin.

### Reagents

All media, serum, antibiotics, and glutamine for cell culture were from Lonza (Basel, Switzerland). Primary antibodies for western blotting were used according to the manufacturer's protocol: poly-(ADPribose)-Polymerase (PARP) (#556494), was purchased from BD Pharmingen™; YAP (#4912) and vimentin (#5741) were purchased from Cell signaling Technology (Danvers, MA, USA); β-Actin (#ab8227), E-cadherin (#40772) and secondary antibodies were purchased as follows: polyclonal goat anti-rabbit IgG (H+L)-HRP conjugate (#1706515) and polyclonal goat anti-mouse IgG (H+L)-HRP conjugate (#1706516) were purchased from Abcam (Cambridge, UK); polyclonal rabbit anti-goat IgG-HRP conjugate (#sc-2768) were purchased from Santa Cruz Biotechnology (Dallas, TX, USA). Goat polyclonal Secondary Antibody to Mouse IgG-H&L-Alexa Fluor® 594 (#ab150120). Microtissues were marked using a green fluorescent probe-cell traker (CellTracker, Promega) and HpaStec with red probe (PKH-26, Sigma Aldrich) according to the manufacturer' protocol. Stem cell viability was evaluated by 3D Cell Viability Assay (ThermoFisher) according to the manufacturer's protocol.

### Drugs

Valproic acid (VPA) was purchased from Enzo Life Sciences (Farmingdale, NY, USA). Simvastatin (#1693), Panobinostat (#1612-25), Atorvastatin (#2278-10) were purchased from Biovision Incorporated (Milpitas, CA, USA). Vorinostat (SAHA) (SML0061) was purchased by Sigma Aldrich, Entinostat (MS-275) (#S1053) was purchased by Selleckchem and Gemcitabine (Accord, Devon, UK) and nab-paclitaxel (Celgene, Milan, Italy) were provided by pharmacy. Recombinant Human TGF-β1 (240-B002) was provided by R&D systems.

### Cell Proliferation Assay and Drugs Combination Studies

Cell proliferation was measured in 96-well plates in cells untreated and treated with described drugs as single agent or in combination. Cell proliferation was measured using a spectrophotometric dye incorporation assay (Sulforhodamine B) an the inhibitory concentration of 50% of cells (IC50) was calculated for each drug, as previously described (Di Gennaro et al Br J Cancer 2010, 103(11):1680-1691). Drugs combination studies were based on concentration-effect curves generated as a plot of the fraction of unaffected (surviving) cells versus drug concentration after 96 h of treatment. Synergism, additivity, and antagonism were quantified after an evaluation of the Combination Index (CI), which was calculated by the Chou-Talalay equation with CalcuSyn software (Biosoft, Cambridge, UK-Chou T C. Cancer Res. 2010 Jan. 15; 70(2):440-6.), as described elsewhere (Terranova-Barberio M, J Exp Clin Cancer Res. 2017; 36(1):177); Bruzzese et al, J Cell Physiol 2011, 226(9):2378-2390). In detail, a CI<0.8, CI<0.9, CI=0.9-1.1, and CI>1.1 indicated a strong synergism, synergysm, additivity or antagonism, respectively, computed at 50%, 75% and 90% of cell kill (respectively ED50, ED75 and ED90) (Terranova-Barberio M, J Exp Clin Cancer Res. 2017; 36(1): 177). The DRI determined the magnitude of dose reduction allowed for each drug when given in combination, compared with the concentration of a single agent that is needed to achieve the same effect. Clonogenic assay Colony formation assay is an in vitro cell survival assay based on the ability of a single cell to grow into a colony. The PANC1, ASPC1 and PANC28 cell lines were plated in 96 well plates with 50 cell/well while the BxPC3 cell line with 100 cell/well. The day after the cells were treated with VPA, SIM alone (at dose of IC10, IC25 and IC50) and in combination. The cells were grown for 10 days. The formed colonies were fixed with TCA (50%) and measured using a spectrophotometric dye incorporation assay (Sulforhodamine B or crystal violet). Microtissue formation assay Pancreatic cancer cell lines PANC1, ASPC1, PANC28, BxPC3 and MiaPaca were cultured as microtissues by the ultra-low attachment (ULA) System (PerkinElmer). The cancer cells were marked using a green fluorescent probe-cell traker (Thermo Fisher) while fibroblast (only for preliminary experiment to evaluate tissues formation) with red probe (PKH-26 Sigma Aldrich) according to manufacture instruction. The 3D microtissue model was obtained using normal fibroblasts or stellate cells isolated from tumor microenvironment of PANC1 xenograft model as scaffold for the PC cell lines in a ratio of 3:1 as described in literature and untreated or treated with drugs, for 96 h with VPA, SIM alone or in combination at the IC25 an IC50. 3D microtissues were maintained in the incubator and photographed by Opera Phenix microscope (Perkin Elmer) air objective magnification 5× and/or scored by Cell Titer-Glo® 3D Cell Viability Assay (Promega) by using a Multimode Reader Cytation 5 (Biotek).

### Western Blotting

Western blots were performed according to standard procedures (Terranova-Barberio et al Oncotarget 2016, 7(7):7715-7731). Images were acquired using the Image Quant LAS 500 and the intensity was measured by Image Quant TL image software (GE Healthcare, Illinois, USA).

### Flow Cytometric Analysis of Apoptotic Cell Death

FACScan flow cytometer analysis was performed on cells treated with VPA and/or SIM at the indicated concentrations, as previously reported (Bruzzese et al Clin Cancer Res 2006, 12(2):617-625). Annexin-V binding was identified by flow cytometry using Annexin-V-FITC staining following the manufacturer's instructions (Becton Dickinson, San Jose, CA).

### RNA Isolation, RT-PCR Assays and Real-Time PCR

RNA was isolated by Trizol reagent (Invitrogen, CA, USA) as previously described (Terranova-Barberio et al Oncotarget 2016, 7(7):7715-7731). Real-Time PCR by ABI Prism 7900 HT Sequence Detection System (Applied Biosystems, CA, USA) was performed using specific primers. All genes relative mRNA expression levels were calculated using the 2-ΔΔCT method and were normalized to that of b-actin as the endogenous control gene β-actin. Probes used were the following: vimentin (QT00004081), ZEB1 (QT00052899), CDH1 (QT00003451), TGFβ (QT00081186), βActin (QT01025850), purchased from Qiagen (Valencia, CA, USA).

### Migration and Invasion Assays

Migration was evaluated by wound-healing assay as previously described (Moreno-Bueno et al, Nat Protoc 2009, 4(11):1591-1613). Briefly, PANC1, PANC28, COLO357, MIAPACA, L3.6pl and ASPC1 cells were seeded to 90% of confluence in 96 well cell carrier ultra (PerkinHelmer). A sterile 10-μl pipette tip was used to longitudinally scratch a constant-diameter stripe in the confluent monolayer to simulate a wound, 24 hours after plating. Then the cells were untreated or exposed to VPA, SIM and gemcitabine/taxol (GEM/TAX) until the wound resulted almost completely closed. At the indicated time wells were photographed by Opera Phenix microscope (PerkinHelmer) air objective magnification 20×. Quantitative measurements were made by determining the distances between the wound-edges in by Harmony software (PerkinHelmer). Invasion assay was performed in transwell, using 8 μm pore size PVPF filters. Briefly, PANC1, PANC28, COLO357, MIAPACA, L3.6pl and ASPC1 5000 cells were seeded in upper part of transwell in medium with FBS 1% after staining with cell tracker green (ThermoFisher) and in the lower part was added 500 μL of medium FBS 10%. After 24 hours the cells are treated and after 48 hours the cells were measured in lower part by Opera Phenix microscope (Perkin Elmer) air objective magnification 5× and counted by Harmony software (Perkin Elmer).

### Immunofluorescence Assay

Cells, plated on slides in 24 wells plate at 50000 cell/well, were treated with drugs as indicated in figure legends. Then cells were fixed in 4% paraformaldehyde (20 min at RT), blocked by 0.2% PBS/BSA solution (5 min at RT) and incubated with primary anti-vimentin antibody for 1 h at 37° C. After washes, cells were incubated with anti-rabbit Alexa Fluor 488 (Thermo Fisher Scientific, Waltham, USA) for 30 min at 37° C. At the indicated time wells were photographed by Opera Phenix microscope (Perkin Elmer) air objective magnification 20×. Quantitative measurements were made by determining the distances between the wound-edges in by Harmony software (Perkin Elmer).

### Cytofluorimetric Assays

PANC1, ASPC1, PANC28 and BxPC3 cell lines were treated as reported in figure legends with VPA and SIM alone or in combination. Cells were collected after 24 h and 48 h and stained with Annexin V-FITC from BD for 15 min at 4° C. for evaluation of apoptotic cells. HPaSteC cells were treated with VPA and SIM alone or in combination and with conditioned medium from PANC1 treated for 24 h with VPA and SIM alone or in combination at the same dosage. Cells were collected after 24 h and stained with GFAP-APC from Thermo Fisher for 30 min at 4° C. for evaluation of activated stellate cells.

### In Vivo Xenograft Studies

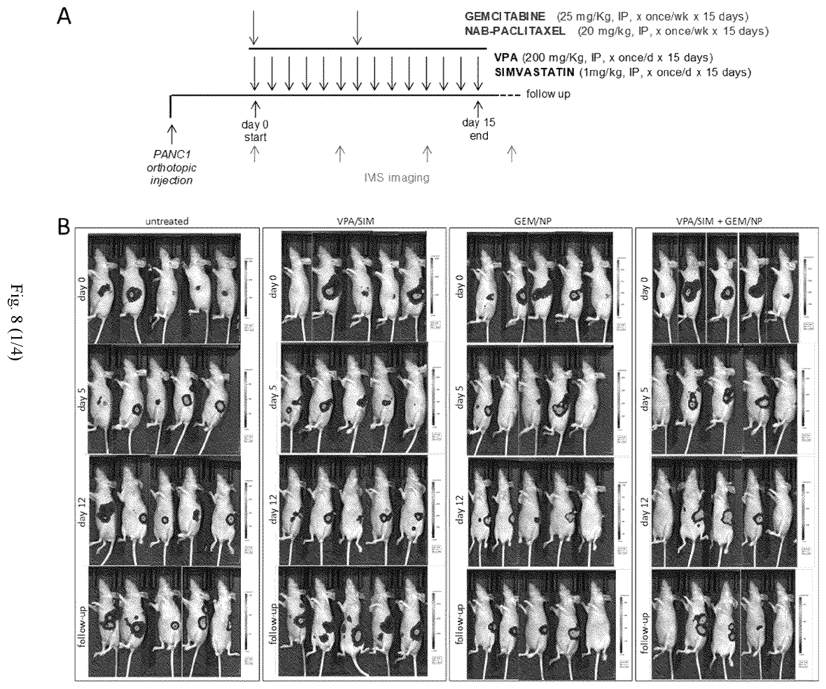

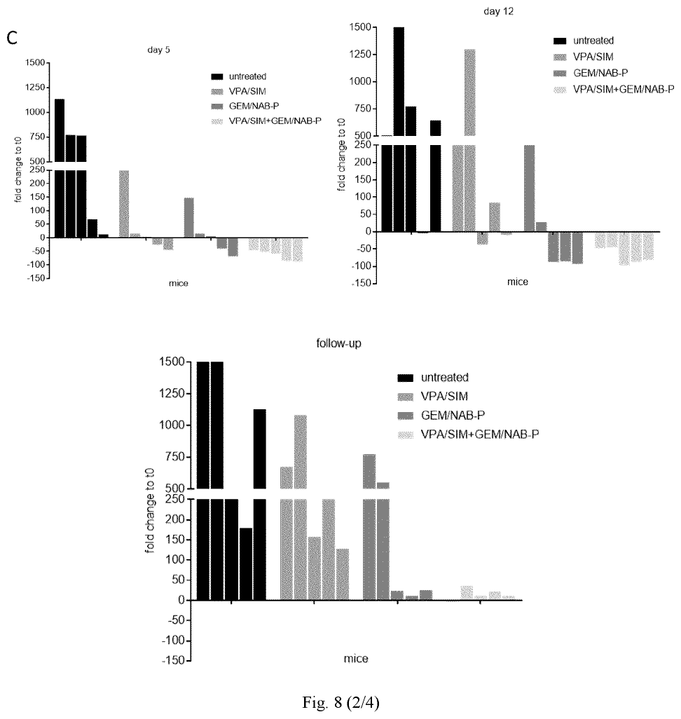

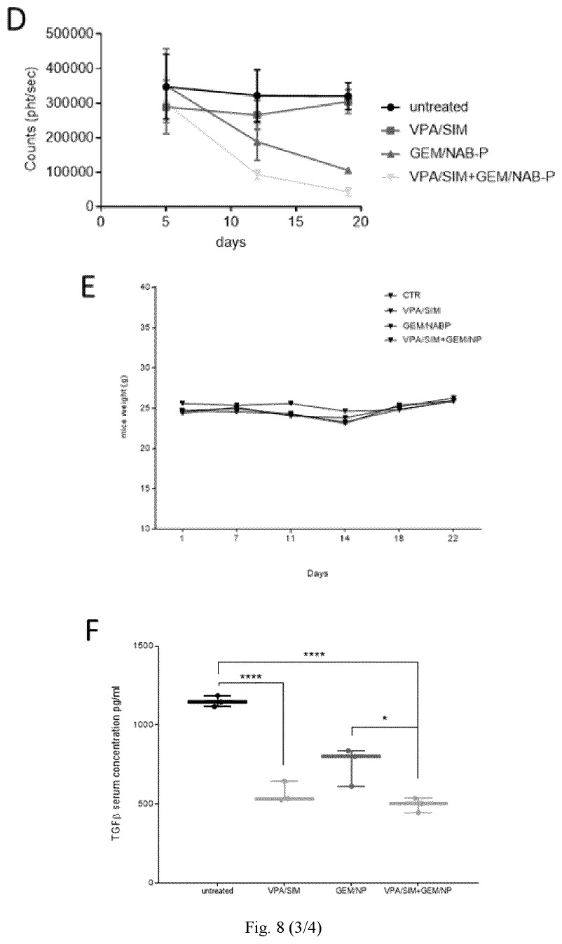

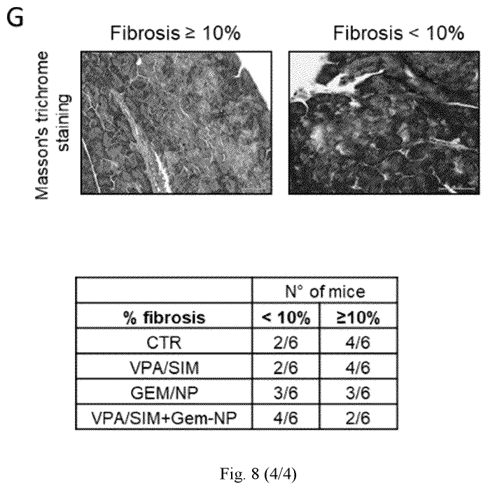

In vivo studies have been performed in accordance with “Directive 2010/63/EU on the protection of Animals used for scientific purposes” and made effective in Italy by the Legislative Decree DLGS 26/2014. For orthotropic xenograft experiment, female athymic nude mice (NCI-nu), which were 6- to 8-weeks old, were purchased from Envigo Laboraties (Huntingdon, UK). The mice were acclimatized in the Animal Care Facility of CROM-Centro Ricerche Oncologiche di Mercogliano. To produce pancreatic tumors, PANC1_LUC cells were harvested from sub-confluent cultures and resuspended in PBS solution. The orthotopic injection of pancreatic cancer cells was performed as described previously (Santoro et al, Mol Cancer Ther 2020, 19(1):247-257). Briefly, the mice were anesthetized with a 3% isoflurane-air mixture. A small incision in the left abdominal flank was made, and the spleen was exteriorized. Tumor cells (0.5×106 240 cells in 50 μL of PBS) were injected subcapsularly in a region of the pancreas just beneath the spleen. A 30-gauge needle, 1 mL disposable syringe were used to inject the tumor cell suspension. A successful subcapsular intrapancreatic injection of tumor cells was identified by the appearance of a fluid bleb without intraperitoneal leakage. One layer of the abdominal wound was closed with wound clips (Auto-clip; Clay Adams, Parsippany, NJ). The mice tolerated the surgical procedure well, and no anesthesia-related deaths occurred. After 1 week, the mice were randomized into four experimental groups (n=5). Mice were treated as followed: (a) vehicles; (b) gemcitabine (weekly 25 mg/Kg, i.p.) and nab-paclitaxel (weekly 20 mg/Kg, i.p.) re-suspended in salt solution 100 μl per dose; (c) VPA (melted in water and diluted in a physiological solution) and SIM (melted in DMSO and diluted in physiological solution); (d) Combination VPA/SIM plus gemcitabine/nab-paclitaxel. Injections were administered for 2 weeks. All mice received drugs vehicles. A schematic representation of treatment was in FIG. 8. The tumor volumes were monitored by IVIS Imaging (PerkinElmer) and the signal intensity (photons/second) was quantified using the Living Image Software 4.1 (PerkinElmer).

For heterotopic xenograft experiment, 2*10⁶ PANC1 cells were suspended in 200 μl of PBS and subcutaneously injected in the right flanks of 6-week-old female balb/c nude mice Envigo Laboraties (Huntingdon, UK). Tumor volume [½ (length× width2)] was assessed using digital caliper 2 times for week (Monday and Thursday). When the tumors became palpable (7 days injection later), the mice were randomized into eight experimental groups (n=6). Mice were treated as followed: (a) vehicles; (b) gemcitabine (weekly 50 mg/Kg, i.p.) and nab-paclitaxel (weekly 10 mg/Kg, i.p.) re-suspended in salt solution 100 μl per dose; (c) valproic acid (200 mg/Kg 7 days/week, per os), simvastatin (2 mg/Kg 7 days/week, per os) re-suspended in salt solution 100 μl per dose; (d-g) double and triple combination. Drug treatments were administered for 22 days. All mice received drugs vehicles. At the end of treatment all mice were sacrificed and tumor samples collected. At the end of treatment (day 28) three mice of each group were scarified and whole blood samples were collected by intracardiac puncture. The blood was centrifuged at 2,500 rpm for 10 min to separate the serum. Biochemistry evaluation of glutamate oxaloacetate transaminase (GOT) activity, glutamate pyruvate transaminase (GPT) activity, and creatinine levels were performed by a COBAS analyzer (Roche).

### TGF-β Blood Mice Quantification

Serum samples were tested using TGF-β1 Quantikine Elisa kit (R&D Systems) following acid activation as indicated in the manufacturer's protocol. A standard curve using 31.5-2,000 μg/ml human recombinant TGF-β1 was generated using the kit reagents and used to calculate the TGF-β1 equivalents in mouse serum. Each specimen was examined in duplicate. Masson's trichrome staining. For direct visualization of collagen fibers and histological assessment of collagen deposition, trichrome staining was performed using the Masson Trichrome Staining Kit (R&D system). A single pathologist (FT) performed a blinded analysis of the slides.

### Statistical Analysis

All experiments were performed at least three times. Statistical significance was determined by the one-way ANOVA Test and a p<0.05 was considered to be statistically significant. All statistical evaluations were performed with Graph Pad Prism 7.

### Results

### Synergistic Effect of Statins/HDACi Combination in PDAC Cell Lines: Repurposing of VPA and Simvastatin as Anticancer Agents.

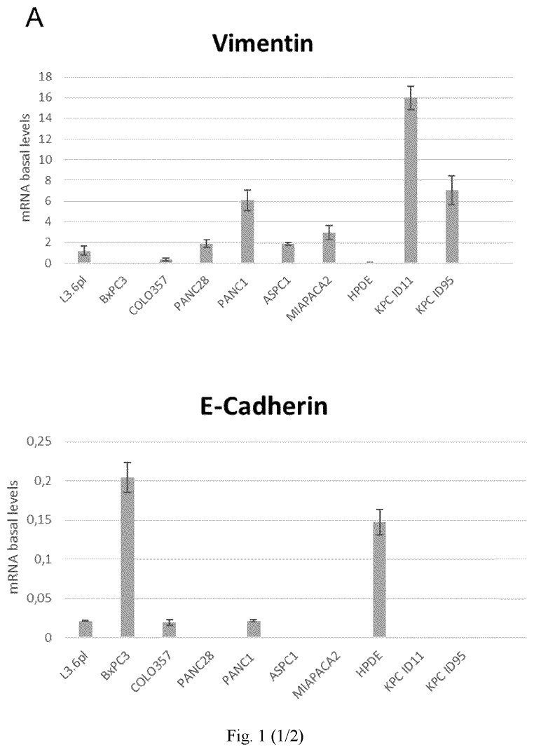

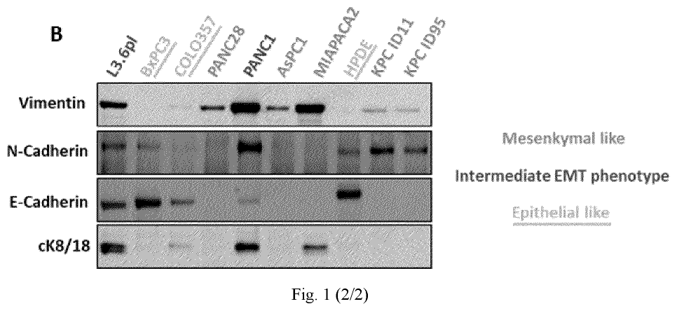

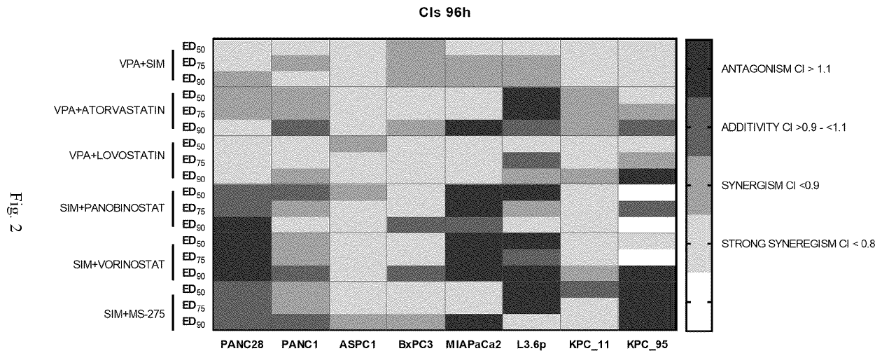

Inventors first evaluated by SRB colorimetric assay the antiproliferative effect of different statins (Simvastatin-SIM, Lovostatin-LOV and Atorvastatin-ATOR) and HDAC inhibitors (Panobinostat-PAN, Vorinostat-VOR, Valproic acid-VPA and Entinostat-MS-275) on a panel of PDAC cell lines with different genetic and phenotypic features (PANC28, PANC1, ASPC1, BxPC3, MIAPACA2, L3.6pl, KPC ID11 AND KPC ID95) (Table 1). Although the cell lines tested showed different basal protein and transcripts levels of epithelial-mesenchymal markers (EMT), at protein and transcript level, (FIG. 1 A-B), they were almost equally sensitive to the anti-proliferative effect of VPA, PAN, VOR and MS-275 at respectively mM, nM, μM concentrations (Table 1).

TABLE 1
Pancreatic cancer cells Models. Anti-proliferative effects of HDAC inhibitors and
Statins evaluated as half maximal inhibitory concentration (IC50) upon 96 hours of treatment.

| | IC₅₀ 96 h mean ± SD | | | | | | |
|---|---|---|---|---|---|---|---|
| | HDAC inhibitors | | | | Statins | | |
| | VPA | PAN | VOR | MS-275 | SIM | ATOR | LOV |
| Cell lines | (mM) | (nM) | (μM) | (μM) | (μM) | (μM) | (μM) |
| MDA-PANC28 | 2.8 ± 0.56 | 22.4 ± 0.56 | 1.6 ± 0.50 | 2.1 ± 0.14 | 1.3 ± 0.07 | 3.25 ± 0.35 | 4.05 ± 0.55 |
| PANC1 | 4.5 ± 0.07 | 13.7 ± 2.75 | 0.82 ± 0.10 | 2.65 ± 0.07 | 9.2 ± 0.90 | 14.4 ± 0.56 | 28.16 ± 5.90 |
| ASPC1 | 3.5 ± 0.07 | 15.6 ± 1.77 | 0.74 ± 0.04 | 2.2 ± 0.14 | 1.5 ± 0.28 | 4.55 ± 0.07 | 8.83 ± 2.40 |
| BxPC3 | 1.82 ± 0.19 | 24.3 ± 5.03 | 1.1 ± 0.30 | 0.9 ± 0.05 | 11.15 ± 2.59 | 16.86 ± 4.30 | 18.66 ± 6.95 |
| COLO357 | 1 ± 0.1 | 65 ± 7.03 | 1 ± 0.20 | >8 | 6 ± 0.5 | 12 ± 2.20 | 8 ± 0.5 |
| MIAPACA | 0.75 ± 0.05 | 16 ± 1.4 | 0.3 ± 0.03 | 0.02 ± 0.12 | 2 ± 0.21 | 1.8 ± 0.06 | 3 ± 0.40 |
| L3.6pl | 2.2 ± 0.12 | >160 | 0.2 ± 0.01 | >8 | 5.1 ± 0.8 | 11.02 ± 3.2 | 12.1 ± 0.33 |
| KPC ID11 | 1.8 ± 0.04 | >160 | 2.5 ± 0.14 | >8 | 2.3 ± 0.48 | 3.1 ± 0.2 | 0.72 ± 0.27 |
| KPC ID95 | 1.6 ± 0.38 | >160 | 1.9 ± 0.15 | >8 | 1.05 ± 0.06 | 4 ± 0.22 | 0.9 ± 0.10 |

Abbreviations:
VPA: Valproic Acid;
SIM: Simvastatin;
PAN: Panobinostat;
VOR: Vorinostat;
MS-275: Entinostat;
ATOR: Atorvastatin;
LOV: Lovastatin.

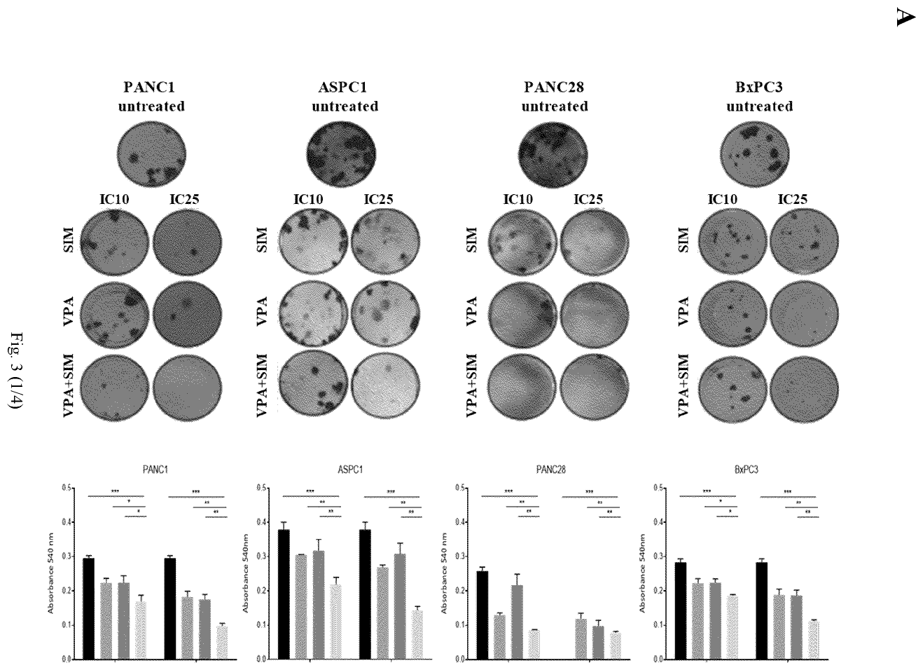

Then, it was performed a systematic screening of the effect of HDACi/Statins combination in the panel of PDAC cell lines, by exploring equipotent doses (50:50 cytotoxic ratio) of the two classes of drugs (FIG. 2 and Table 1). Good results were obtained with consistent antitumor synergistic effects with low combination indexes (CIs), calculated at 50% (CI50), 75% (CI75) and 90% (CI90) of cell lethality in all cell lines when VPA was associated with either SIM, LOV or ATOR. On the other hands among the other HDACIs tested, VPA in combination with SIM seems to be the best one in all PDAC cells tested. Thus, for all further experiments inventors focused their attention on VPA/SIM combination.

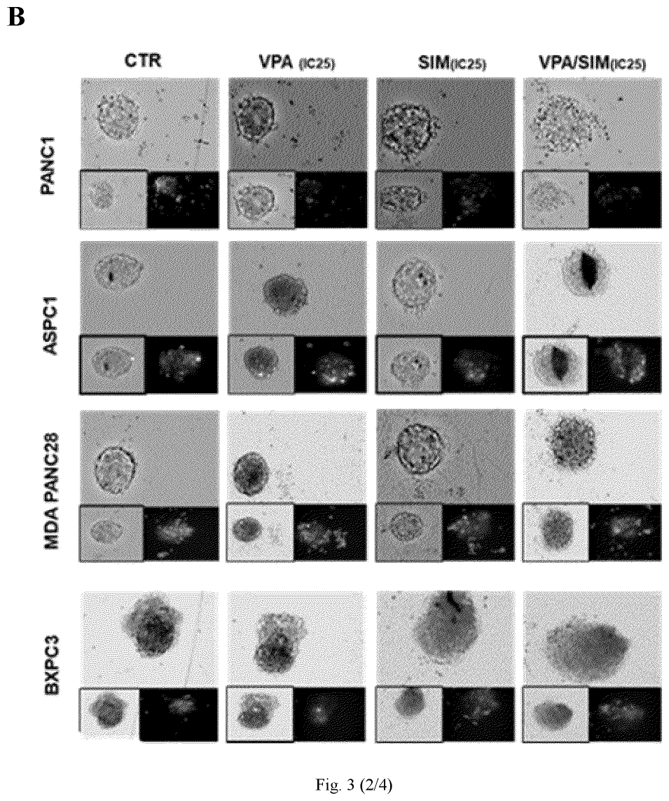

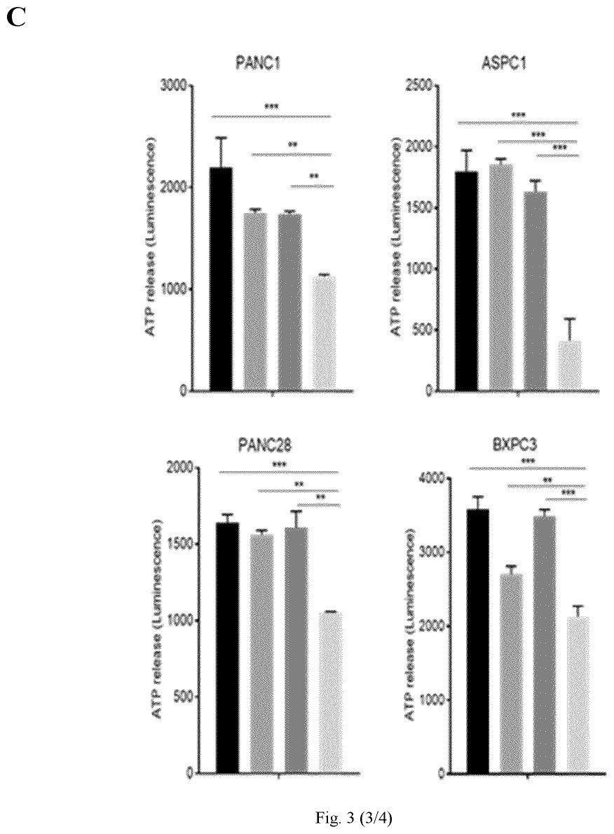

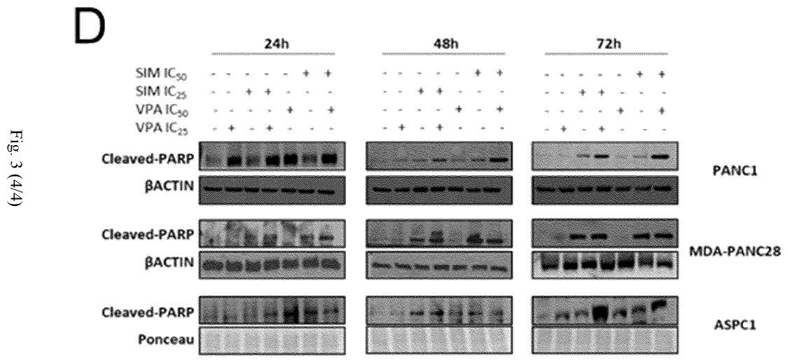

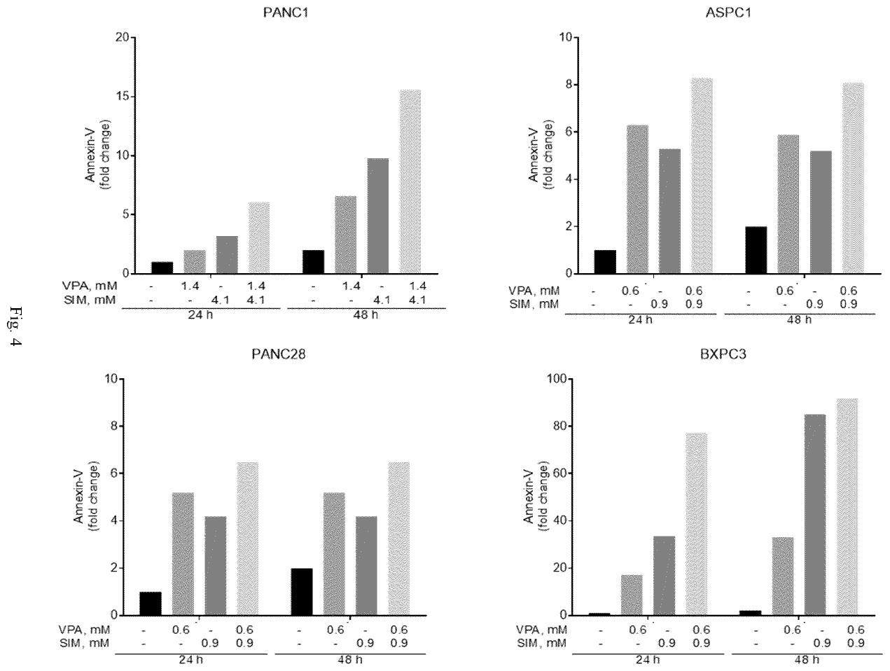

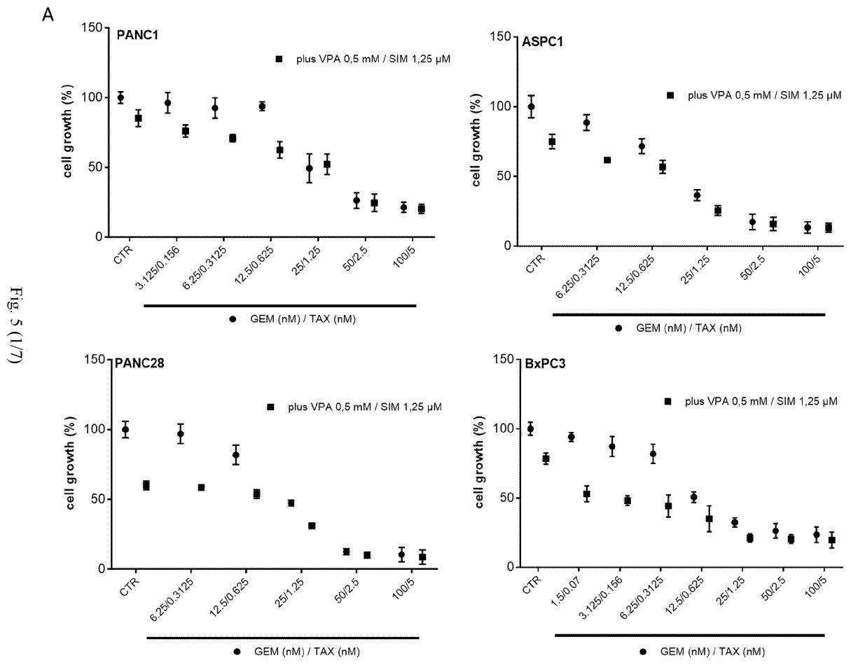

The synergistic effect observed thus far was next evaluated, with a colony formation assay, to determine the effect of VPA and SIM alone or in combination at high (IC25) and low (IC10) doses, on PANC1, ASPC1, PANC28 and BxPC3 cells (FIG. 3A). In all cell lines, VPA and SIM alone or in combination inhibited colonies formation and, when the two drugs were administered together, a statistically significant potentiation of the anti-proliferative effect of the drugs also at low doses was observed (FIG. 3A). Since 3D cultures better recapitulate tumour growth complexity compared to 2D monolayers conditions, inventors tested the combinations also on 3D cultures from PANC1, ASPC1, PANC28 and BxPC3 cells. It was used the Cell Carrier Spheroid ULA microplates that enable the use of 3D cell culture models for cell-based microplate assays and forms consistently round spheroids for highly reproducible imaging data. The 3D microtissue model was obtained using normal fibroblasts as scaffold for the PDAC cell lines in a ratio of 3:1 as described in literature [Brancato V, et al., Biomaterials 2020, 232:119744.]. Microtissues were obtained after 24 h and the confocal images showed a strong interaction among fibroblasts and PDAC cells with the clear presence of a membrane around the cells aggregate. The assembled microtissues were treated for 96 h with VPA and SIM alone or in combination (IC25 for each drug). The data showed that VPA/SIM combination leads to an almost completely disruption of microtissues compared to control or single treatment (FIG. 3B), confirmed by the ATP bioluminescence assay on microtissues that showed a statistically significant reduction in cell viability in combination treatments, in all cell models analyzed (FIG. 3C).

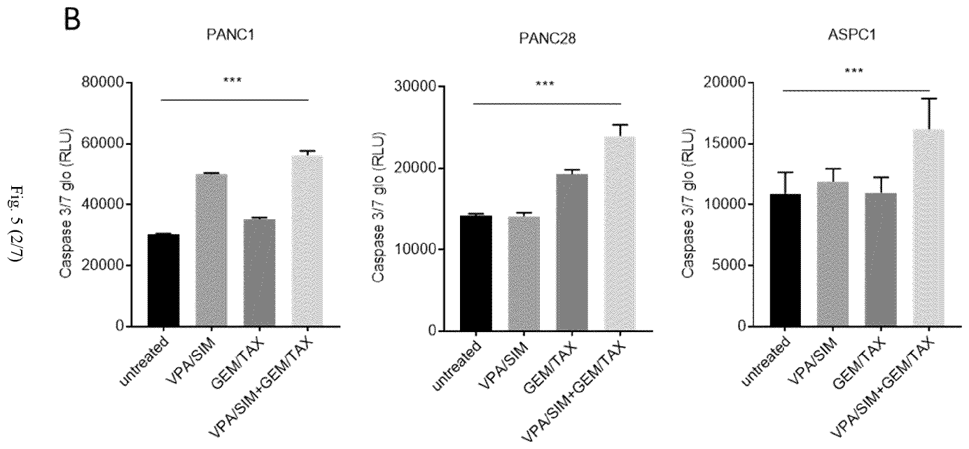

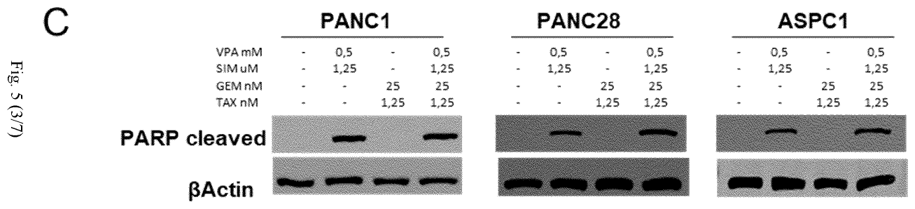

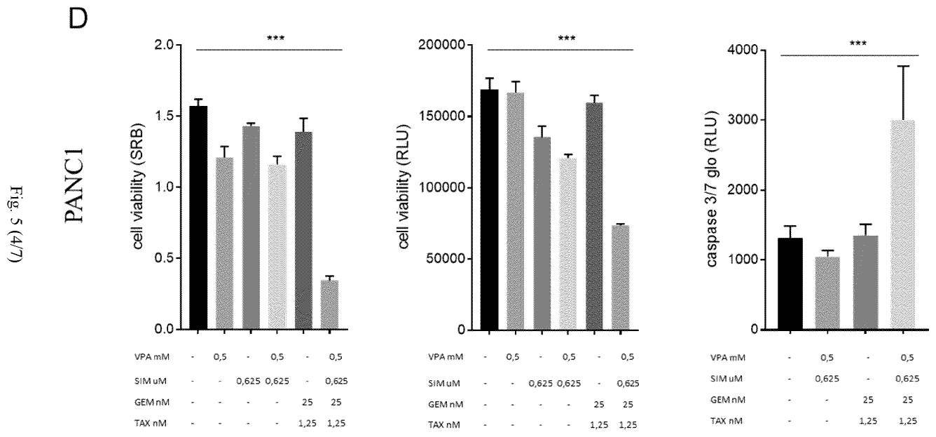

Consistently with the data reported above, a significant induction of apoptosis was found in all PDAC cells treated with VPA/SIM combination as showed by a clear PARP cleavage after 24 and 48 h of treatment especially at the IC50 of the two drugs (FIG. 3D). These results were confirmed by cytofluorimetric evaluation of annexin V positivity cells at 48 and 72 h (FIG. 4).

### Combination of VPA/SIM Plus Gemcitabine/Taxol Induces Apoptosis and Reduces Cell Growth in Monolayer and Spheroids Culture in PDAC Cells.

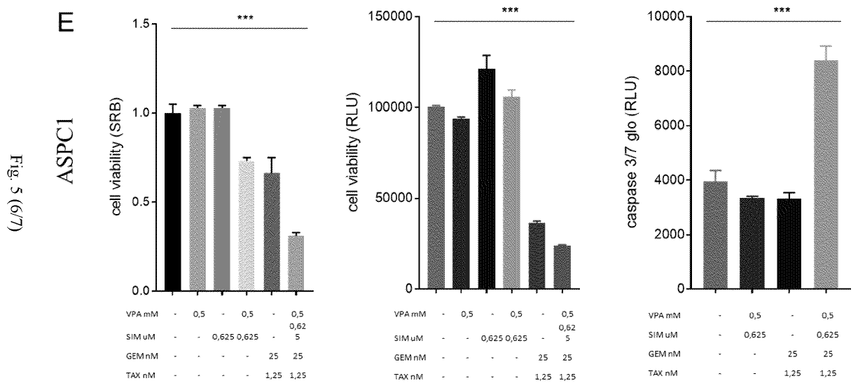

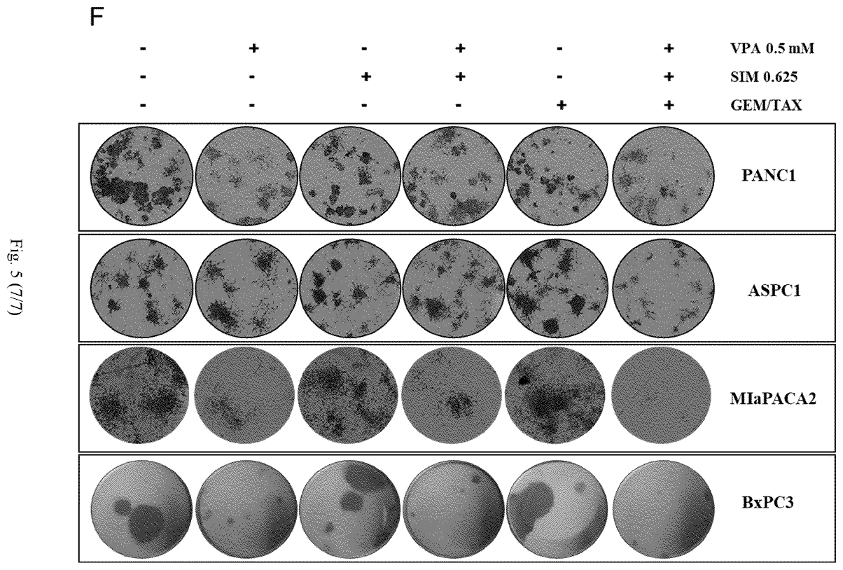

Inventors next explored the potential of VPA/SIM combination to sensitize PDAC cells to gemcitabine/taxol (GEM/TAX) chemotherapy doublet. The combination of VPA/SIM at fixed low doses (VPA 0.5 mM and SIM 0,625 μM) given simultaneously to increasing concentrations of GEM/TAX was investigated (FIG. 5A). A consistent synergistic anti-proliferative effects was obtained when low doses of VPA/SIM were combined with the lowest concentration of GEM/TAX in all the four PDAC cell lines tested, compared with dual combinations GEM/TAX alone. Interestingly, these results were validated in primary PDAC cells (data not showed). Furthermore, a synergistic induction of apoptosis was showed in PANC1, PANC28 and ASPC1, measure as caspase 3/7 activation (FIG. 5B), and PARP cleavage (FIG. 5C), by the VPA/SIM/GEM/TAX combination vs doublet combinations. In order to verify the efficacy of this combination in a more complex model that better recapitulate tumor growth, inventors took advantage of 3D cell self-assembled spheroid system. In detail, the cells were plated in low-attached plate in sphere medium to form 1st generation sphere and after 48 h the cells were treated for 24 hours with VPA and SIM. Then the spheroids were disaggregated and plated again to obtain a second generation in three different condition to highlight different effects. In FIGS. 5D and 5E, PANC1 and ASPC1 were plated, respectively, the second generation in adherent condition, and treated them as indicated for 96 h, to investigate the impact of treatment on the viability of cells with more aggressive features (FIG. 5D-E graphs on the right). Next, inventors evaluate the capacity of treatment to prevent/reduce more aggressive tumors in terms of viability (FIG. 5D-E graphs in the middle) and in terms of induction of apoptosis (FIG. 5D-E graphs on the left), seeding the second generation in 3D system in the presence of drugs for 72 h and 24 h, respectively. All 3D cultures experiments confirmed a strong inhibitory effect of VPA/SIM combination and a clear potentiation of GEM/TAX effect, which, as single treatment, was poorly effective. In line, we demonstrated in data not shown, the efficacy of VPA/SIM combination to sensitize to GEM/TAX treatment microtissues generated co-culturing PANC1 with stellate cells isolated from PANC1-xenograft model tumor microenvironment. Finally, VPA and SIM alone or in combination with chemotherapy inhibited colonies formation and, when the four drugs were administered together, we observed a significant potentiation of the anti-proliferative effect of the drugs also at low doses (FIG. 5F).

Valproic Acid and Simvastatin in Combination with Gemcitabine/Taxol Target TGFβ-Induced EMT.

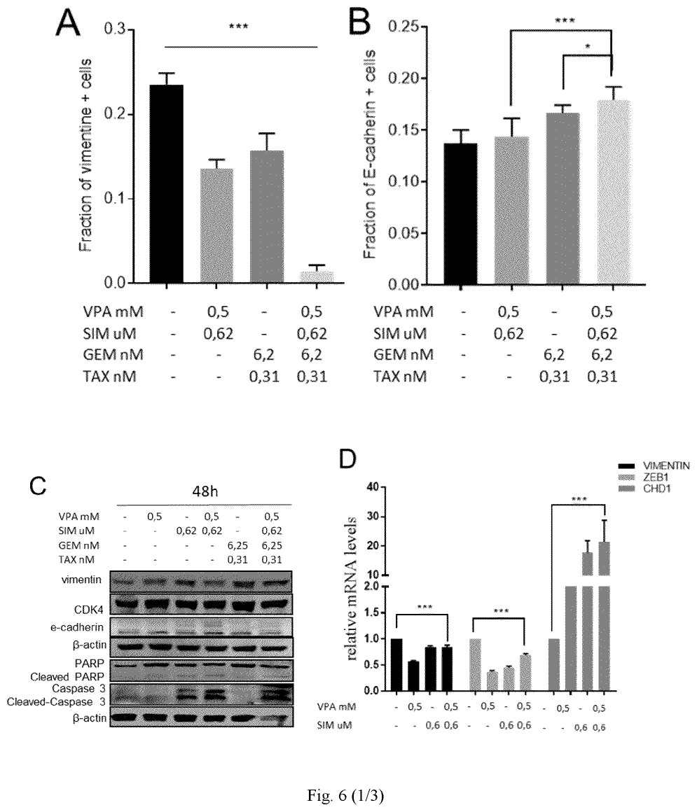

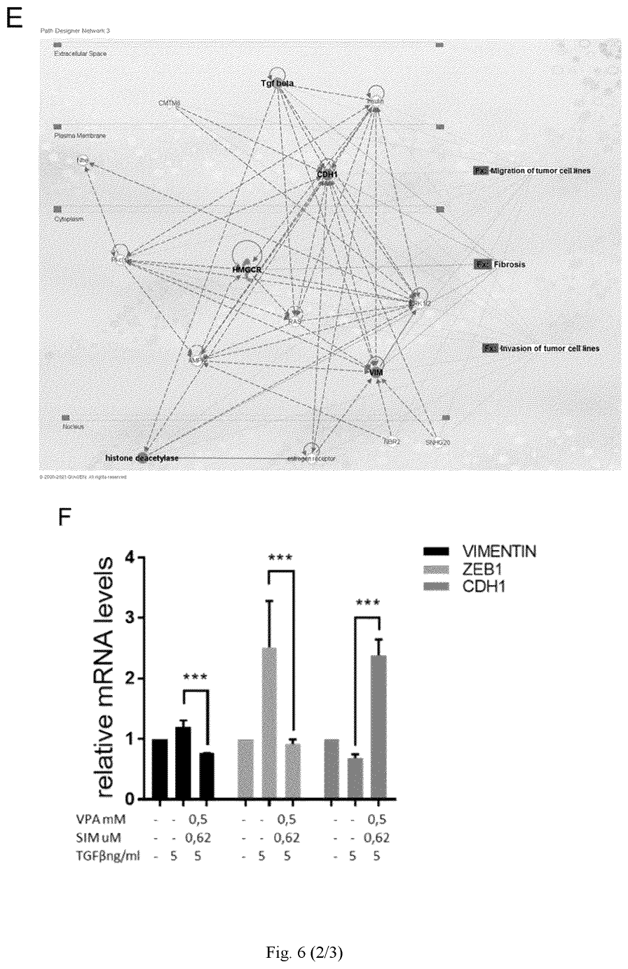

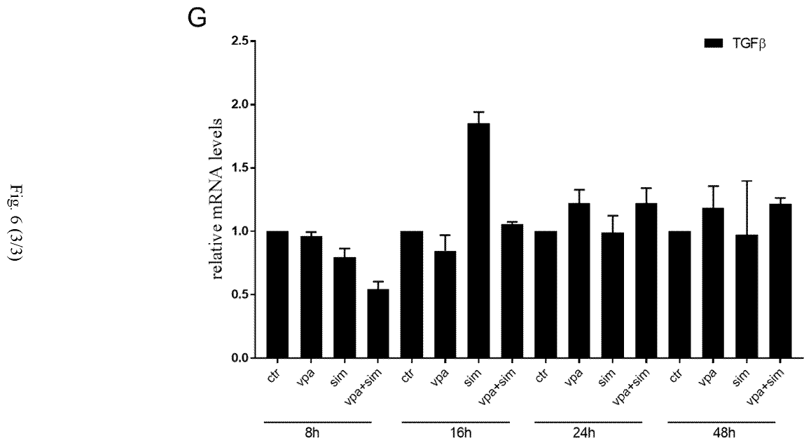

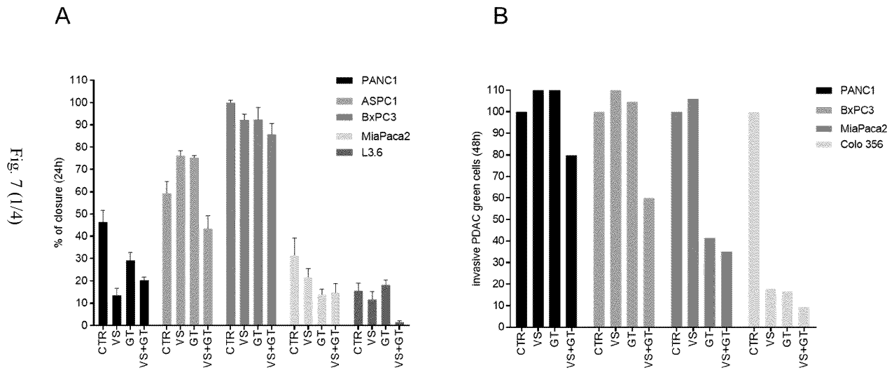

Several evidences demonstrated that the engagement of EMT program renders PDAC cells more invasive and resistant to therapy-induced apoptosis. Here, inventors showed that VPA/SIM combination led to changes in EMT-markers with an increase in e-cadherin and a decrease in vimentin protein levels. This effect was maintained or further enhanced in combination with GEM/TAX, along with an induction of proapoptotic effect evaluated by PARP and Caspase 3 cleavage (FIG. 6A-B-C). Consistently, inventors also observed a similar decrease in the mRNA levels of mesenchymal markers vimentin and of e-cadherin-repressor Zinc Finger E-Box Binding Homeobox 1 (ZEB1), paralleled by an increase in e-cadherin (CDH1) mRNA levels (FIG. 6D). To further disclose the molecular mechanism behind the ability of VPA/SIM combination to synergistically interact with GEM/TAX involving EMT modulation, we performed an ingenuity pathway analysis (IPA) search on “vimentin and e-cadherin” combined with “HDACs and HMGCR”, the targets of VPA and SIM, respectively. As shown in FIG. 6E we revealed a network with direct and indirect relationships connecting all the protein used as input, confirming a functional relationship between the targets of our treatment combination and the EMT markers. The transforming growth factor β (TGFβ), came out in this IPA network as a central hierarchical dominant hub, in line with abundant literature that defines TGFβ as one of the primary drivers of EMT, particularly in PDAC. Consistently, “Migration of tumor cell lines”, “Fibrosis” and “Invasion of tumor cell lines” were the most significantly enriched functions for this network (FIG. 6E). These results led us to deepen this relationship testing the hypothesis that VPA/SIM synergistically with GEM/TAX combination could attenuates TGFβ-induced EMT. Moreover, inventors confirmed the ability of VPA/SIM to prevents TGFβ-induced modulation of EMT markers (VIM, ZEB1 and CDH1) in PANC1 cells following TGFβ stimulation (FIG. 6F). They have also evaluated the ability of VPA/SIM to directly affect TGFβ at transcriptional level, showing that VPA/SIM significantly attenuated its transcription at very early time point (FIG. 6G).

VPA/SIM Synergistically with Gemcitabine/Taxol Reduces PANC1 Cell Migration and Invasion Capability and Impacts on PDAC Microenvironment.

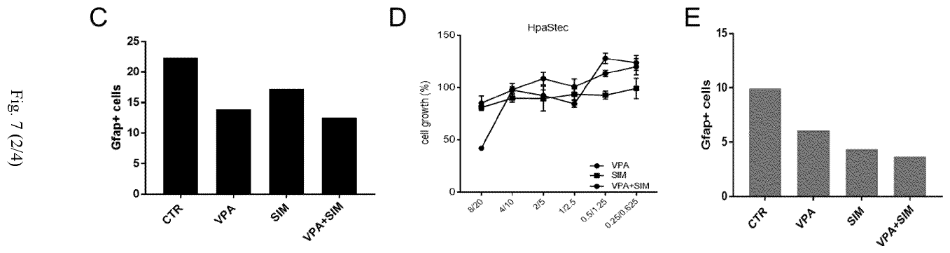

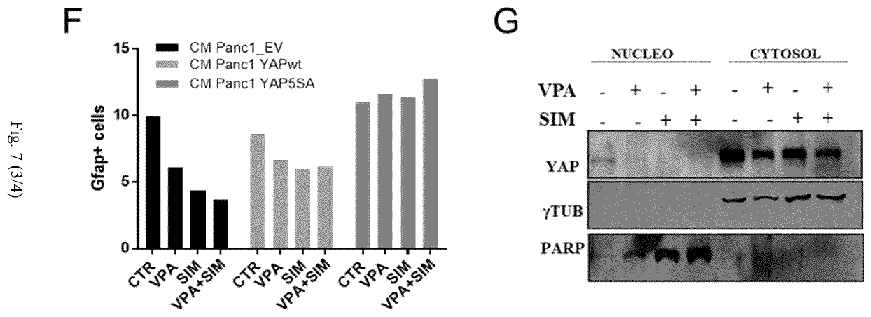

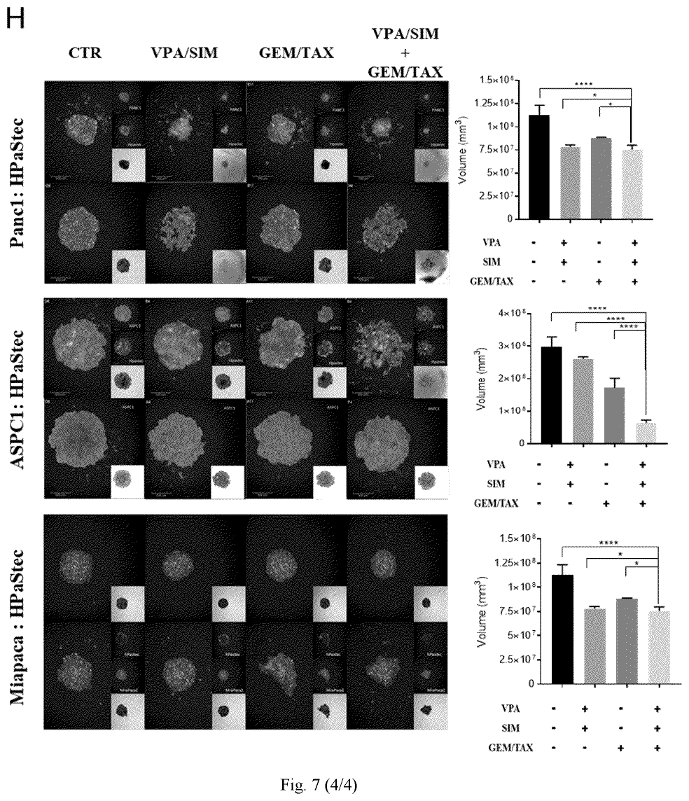

The effects mechanistically evaluated were functionally tested evaluating the ability of the present combination to affect the ability of cells to migrate and to invade. This demonstrated that the VPA/SIM in combination with GEM/TAX markedly inhibited the migration and invasion in a panel of PDAC cell lines (FIG. 7A-B).

Several published evidence have shown that activated stellate cells, a fibroblast population resident in pancreatic stroma, play a critical role in the pathogenesis of pancreatic fibrosis (desmoplasia) and pancreatitis (Thomas D. et al, Molecular Cancer 2019). Since that these cells, once activated by tumor cells crosstalking, can regulate ECM remodeling leading to chemoresistance, inventors decided to test the ability of VPA/SIM combination to interfere with their activation, directly and by modulating cancer cells/fibroblasts crosstalk. At first HPaSteC cells were treated with VPA and/or SIM for 24 h and showed the ability of the treatment to impair their activation measured as G-FAP positive cells (FIG. 7C), without affecting their proliferation status (FIG. 7D). Interestingly, similar results were obtained also treating the HpaSteC with media conditioned by a PDAC cell line, PANC1, untreated or treated with VPA/SIM (FIG. 7E), suggesting the ability of our treatment to interfere with the crosstalk between PDAC and HpaStec cells.

To go in deep in the mechanism, it was found that the VPA/SIM inhibitory effects, described above, was reverted by the use of conditioned medium from PANC1 cells transfected with constitutive active YAP oncogene (YAP5SA), confirming its involvement in this mechanism (FIG. 7F), along with the impaired YAP translocation in to the nucleus upon VPA/SIM treatment (FIG. 7G).

In line, inventors demonstrated the efficacy of VPA/SIM combination to sensitize to GEM/TAX treatment microtissues generated co-culturing PANC1, ASPC1 and MIAPACA2 cells with human stellate cells HpaSteC. This synergistic antitumor effect was sharply clear in PANC1 and ASPC1 cells and slightly weaker in MIAPACA cells, where microtissues appeared reduced, in terms of volume, upon the treatment with all the combinations almost at the same level (FIG. 7G).

In Vivo Synergistic Antitumor Effect of Valproic Acid and Simvastatin in Combination with Gemcitabine/Nab-Paclitaxel

The synergistic interaction of the proposed combination was confirmed in both orthotropic and heterotopic xenograft in vivo model. For orthotropic model, which better recapitulate the PDAC tumor microenvironment, inventors took advantage of PANC1_LUC cells injected into the pancreatic gland of mice. One week after implantation the mice were randomly assigned to receive subtherapeutic doses of VPA/SIM combination (200 mg/Kg and 2 mg/Kg, respectively, i.p. daily for 2 weeks), and GEM/NP (Gemcitabine weekly 25 mg/Kg, i.p. and nab-Paclitaxel weekly 20 mg/Kg, i.p.); the combination VPA/SIM+GEM/NP, or their vehicles as schematized in FIG. 8A. Tumor growth was monitored once a week by photon intensity at the specified time point (FIG. 8B) and for each group, the measurements of single mouse at day 5, day 12 and after one week from the end of treatment (follow up), were compared with the respective values at day 1 (T0) (FIG. 8C). A clear tumor growth inhibition was already observed within 5 days from the start of treatment, for all the mice in VPA/SIM+GEM/NP group as compared with the other groups, and maintained over time (FIG. 8C). VPA/SIM+GEM/NP combination produced a statistically significant tumor growth inhibition compared with control and single treatments groups, evaluated as means of the measurements for each group (FIG. 8D). The combined treatment was well tolerated by xenografted mice, as shown by the maintenance of body weight (FIG. 8E) and by the absence of other signs of acute or delayed toxicity. Consistently with the in vitro results, a significant reduction of circulating TGFβ1 levels was obtained in serum from mice treated with VPA/SIM in combination with GEM/NP (FIG. 8F) paralleled by a reduced fibrosis, assessed by the Masson's trichromatic staining on pancreatic tumor sections. In detail four out of six mice/group treated with the combination VPA/SIM plus GEM/NP showed <10% of fibrosis, instead the bulk of mice in the control showed ≥10% of fibrotic areas (FIG. 8G). Consistently, data not shown demonstrated the capability of VPA/SIM combination to target pancreatic stellate cells by impairing the levels of their main activation marker GPAF.

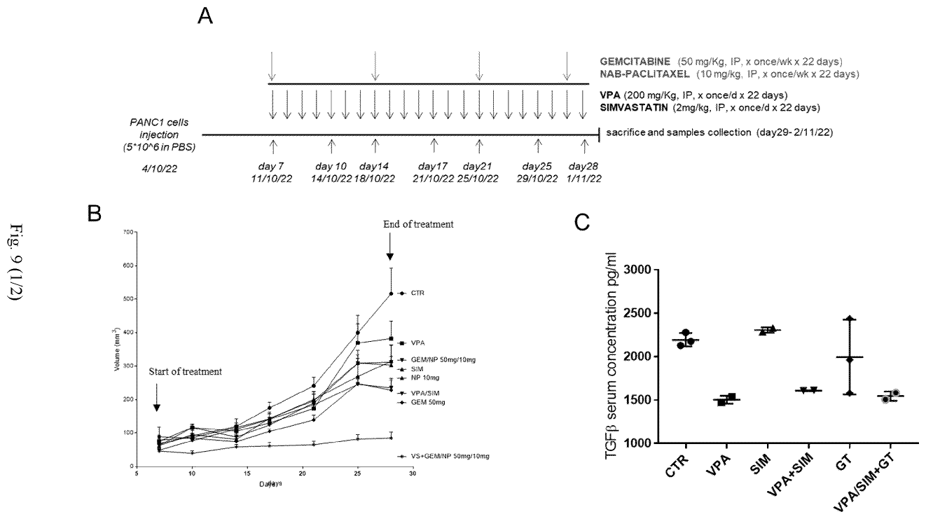

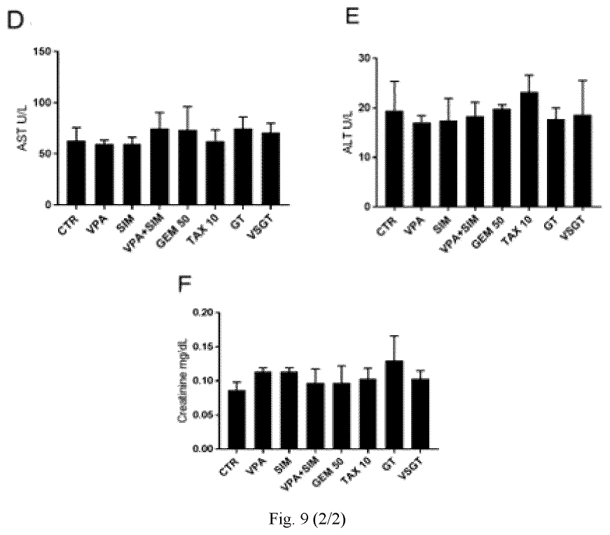

For heterotopic model, PANC1 cells were injected in the right flank of mice (2*10⁶). One week after implantation the mice were randomly assigned to receive VPA/SIM combination (200 mg/Kg and 2 mg/Kg, respectively, i.p. daily for 2 weeks), and GEM/NP (Gemcitabine weekly 50 mg/Kg, i.p. and Nab-Paclitaxel weekly 10 mg/Kg, i.p.) and combination, this time, exploring a ratio of GEM/NP more comparable to those employed in clinical practice to treat PDAC patients, as reported in schematic representation (FIG. 9A). The tumor volume was measured by caliper (FIG. 9B). A significant reduction of circulating TGF-1 levels in mice treated with VPA/SIM in combination with GEM/NP was obtained also in this model (FIG. 9C). Furthermore, no significant changes of the biochemical parameters (GOT, GPT, and creatinine) were reported in serum of untreated mice compared with treated groups, confirming that the combination did not increase significantly the liver toxicity of the single treatments (FIG. 9E-F-G).

1. A method for the treatment of pancreatic cancer, comprising administering an HDAC inhibitor and a statin to a subject in need thereof

2. The method according to claim 1, wherein the HDAC inhibitor is selected from valproic acid or a salt thereof, panobinostat, vorinostat, entinostat or mocetinostat, and the statin is selected from simvastatin, atorvastatin, or lovastatin.

3. The method according to claim 2, wherein the HDAC inhibitor is valproic acid and the statin is simvastatin.

4. The method of claim 1, further comprising administering at least one further anti-cancer agent.

5. The method of claim 1, wherein the subject has responded to, or is resistant to, or has developed resistance to a first line therapy.

6. The method according to claim 5, wherein the first line therapy comprises administration of gemcitabine and/or nab-paclitaxel.

7. The method of claim 1, wherein the ratio of the HDAC inhibitor and the statin is of about 50:50 cytotoxic ratio.

8. The method of claim 1, wherein the HDAC inhibitor is selected from:
valproic acid at a concentration ranging from about 16 mm to about 0.5 mM; and/or
vorinostat at a concentration ranging from about 16 μM to about 0.125 μM; and/or
panobinostat at a concentration ranging from about 160 nM to about 2.5 nM; and/or
entinostat at a concentration ranging from about 16 μM to about 0.125 μM;
and wherein the statin is selected from:
simvastatin at a concentration ranging from about 8 μM to about 0.06 μM; and/or
atorvastatin at a concentration ranging from about 8 μM to about 0.06 μM; and/or
lovastatin at a concentration ranging from about 8 μM to about 0.06 μM.

9. The method of claim 4, wherein the further anti-cancer agent is selected from one or more of a Btk tyrosine kinase inhibitor, an Erbb2 tyrosine kinase receptor inhibitor; an Erbb4 tyrosine kinase receptor inhibitor, an mTOR inhibitor, a thymidylate synthase inhibitor, an EGFR tyrosine kinase receptor inhibitor, an Epidermal growth factor antagonist, a Fyn tyrosine kinase inhibitor, a kit tyrosine kinase inhibitor, a Lyn tyrosine kinase inhibitor, a NK cell receptor modulator, a PDGF receptor antagonist, a PARP inhibitor, a poly ADP ribose polymerase inhibitor, a poly ADP ribose polymerase 1 inhibitor, a poly ADP ribose polymerase 2 inhibitor, a poly ADP ribose polymerase 3 inhibitor, a galactosyltransferase modulator, a dihydropyrimidine dehydrogenase inhibitor, an orotate phosphoribosyltransferase inhibitor, a telomerase modulator, a mucin inhibitor, a secretin agonist, a TNF related apoptosis inducing ligand modulator, an IL17 gene stimulator, an interleukin 17E ligand, a Neurokinin receptor agonist, a cyclin G1 inhibitor, a checkpoint inhibitor, a PD-1 inhibitor, a PD-L1 inhibitor, a CTLA4 inhibitor, a topoisomerase I inhibitor, an Alk-5 protein kinase inhibitor, a connective tissue growth factor ligand inhibitor, a notch-2 receptor antagonist, a notch-3 receptor antagonist, a hyaluronidase stimulator, a MEK-1 protein kinase inhibitor; MEK-2 protein kinase inhibitor, a GM-CSF receptor modulator; TNF alpha ligand modulator, a mesothelin modulator, an asparaginase stimulator, a caspase-3 stimulator; caspase-9 stimulator, a PKN3 gene inhibitor, a hedgehog protein inhibitor; Smoothened receptor antagonist, an AKT1 gene inhibitor, a DHFR inhibitor, a thymidine kinase stimulator, a CD29 modulator, a fibronectin modulator, an interleukin-2 ligand, a serine protease inhibitor, a D40LG gene stimulator; TNFSF9 gene stimulator, a 2-oxoglutarate dehydrogenase inhibitor, a TGF-beta type II receptor antagonist, an Erbb3 tyrosine kinase receptor inhibitor, a cholecystokinin CCK2 receptor antagonist, a Wilms tumor protein modulator, a Ras GTPase modulator, a cyclin-dependent kinase 4 inhibitor A modulator, an estrogen receptor beta modulator, a 4-1BB inhibitor, a 4-1BBL inhibitor, a PD-L2 inhibitor, a B7-H3 inhibitor, a B7-H4 inhibitor, a BTLA inhibitor, a HVEM inhibitor, aTIM3 inhibitor, a GAL9 inhibitor, a LAG3 inhibitor, a VISTA inhibitor, a KIR inhibitor, a 2B4 inhibitor, a CD160 inhibitor and a CD66e modulator, a taxane or a combination thereof.

10. The method of claim 4, wherein the further anti-cancer agent is selected from bavituximab, IMM-101, CAP1-6D, Rexin-G, genistein, CVac, MM-D37K, PCI-27483, TG-01, LOAd-703, CPI-613, upamostat, CRS-207, NovaCaps, trametinib, Atu-027, sonidegib, GRASPA, trabedersen, nastorazepide, Vaccell, oregovomab, istiratumab, refametinib, regorafenib, lapatinib, selumetinib, rucaparib, pelareorep, tarextumab, PEGylated hyaluronidase, varlitinib, aglatimagene besadenovec, GBS-01, GI-4000, WF-10, galunisertib, afatinib, RX-0201, FG-3019, pertuzumab, DCVax-Direct, selinexor, glufosfamide, virulizin, yttrium (90Y) clivatuzumab tetraxetan, brivudine, nimotuzumab, algenpantucel-L, tegafur+gimeracil+oteracil potassium+calcium folinate, olaparib, ibrutinib, pirarubicin, Rh-Apo2L, tertomotide, tegafur+gimeracil+oteracil potassium, tegafur+gimeracil+oteracil potassium, masitinib, Rexin-G, mitomycin, erlotinib, adriamycin, dexamethasone, vincristine, cyclophosphamide, topotecan, taxol, interferons, platinum derivatives, taxane, paclitaxel, vinca alkaloids, vinblastine, anthracyclines, doxorubicin, epipodophyllotoxins, etoposide, cisplatin, rapamycin, methotrexate, actinomycin D, dolastatin 10, colchicine, emetine, trimetrexate, metoprine, cyclosporine, daunorubicin, teniposide, amphotericin, alkylating agents, chlorambucil, 5-fluorouracil, campthothecin, metronidazole, Gleevec, panitumumab, abarelix, aldesleukin, alemtuzumab, alitretinoin, allopurinol, altretamine, amifostine, anastrozole, arsenic trioxide, asparaginase, azacitidine, AZD9291, BCG Live, bevacuzimab, bexarotene, bleomycin, bortezomib, busulfan, calusterone, capecitabine, camptothecin, carboplatin, carmustine, celecoxib, cetuximab, chlorambucil, cladribine, clofarabine, cyclophosphamide, cytarabine, dactinomycin, darbepoetin alfa, daunorubicin, denileukin, dexrazoxane, docetaxel, doxorubicin (neutral), doxorubicin hydrochloride, dromostanolone propionate, epirubicin, epoetin alfa, estramustine, etoposide phosphate, etoposide, exemestane, filgrastim, floxuridine fludarabine, fulvestrant, gefitinib, gemcitabine, gemtuzumab, goserelin acetate, histrelin acetate, hydroxyurea, ibritumomab, idarubicin, ifosfamide, imatinib mesylate, interferon alfa-2a, interferon alfa-2b, irinotecan, lenalidomide, letrozole, leucovorin, leuprolide acetate, levamisole, lomustine, megestrol acetate, melphalan, mercaptopurine, 6-MP, mesna, methotrexate, methoxsalen, mitomycin C, mitotane, mitoxantrone, nandrolone, nelarabine, nofetumomab, oprelvekin, oxaliplatin, paclitaxel, palifermin, pamidronate, pegademase, pegaspargase, pegfilgrastim, pemetrexed disodium, pentostatin, pipobroman, plicamycin, porfimer sodium, procarbazine, quinacrine, rasburicase, rituximab, rociletinib, sargramostim, sorafenib, streptozocin, sunitinib maleate, talc, tamoxifen, temozolomide, teniposide, VM-26, testolactone, thioguanine, 6-TG, thiotepa, topotecan, toremifene, tositumomab, trastuzumab, tretinoin, ATRA, uracil mustard, valrubicin, vinblastine, vincristine, vinorelbine, zoledronate, zoledronic acid, pembrolizumab, nivolumab, IBI-308, mDX-400, BGB-108, MEDI-0680, SHR-1210, PF-06801591, PDR-001, GB-226, STI-1110, durvalumab, atezolizumab, avelumab, BMS-936559, ALN-PDL, TSR-042, KD-033, CA-170, STI-1014, FOLFIRINOX and KY-1003, and combinations thereof.

11. The method of claim 4, wherein the further anti-cancer agent is taxol, gemcitabine, nab-paclitaxel, cisplatin, capecitabine, irinotecan or a combination thereof.

12. The method according to claim 11, wherein the further anticancer agent is a combination of taxol and gemcitabine or a combination of nab-paclitaxel and gemcitabine or a combination of gemcitabine, nab-paclitaxel, cisplatin, and capecitabine.

13. The method of claim 1, wherein the pancreatic cancer is selected from the group consisting of pancreatic adenocarcinoma, non-resectable pancreatic cancer, locally advanced pancreatic cancer, borderline resectable pancreatic cancer, locally advanced pancreatic ductal adenocarcinoma, borderline resectable pancreatic ductal adenocarcinoma, metastatic pancreatic cancer, chemotherapy-resistant pancreatic cancer, pancreatic ductal adenocarcinoma, squamous pancreatic cancer, pancreatic progenitor, immunogenic pancreatic cancer, aberrantly differentiated endocrine exocrine (ADEX) tumors, an exocrine pancreatic cancer, pancreatic intraepithelial neoplasia, intraductal papillary mucinous neoplasms, mucinous cystic neoplasms, mucinous pancreas cancer, adenosquamous carcinoma, signet ring cell carcinoma, hepatoid carcinoma, colloid carcinoma, undifferentiated carcinoma, undifferentiated carcinomas with osteoclast-like giant cells, a pancreatic cystic neoplasm, an islet cell tumor, a pancreas endrocrine tumor, and a pancreatic neuroendrocrine tumor.

14. The method of claim 1, wherein the HDAC inhibitor and the statin are administered in a single dosage unit or separately.

15. The method according to claim 14, wherein the single dosage unit comprises at least one pharmaceutically acceptable excipient.

16. The method according to claim 14, wherein the single dosage unit or the separate dosage formulations are in the form of an oral, parenteral and/or topical dosage forms.
